(19) United States
(12) Patent Application Publication
(10) Pub. No.: US 2012/0296362 A1
(43) Pub. Date: Nov. 22, 2012

(54) VASCULAR REMODELING DEVICE

(75) Inventors: Anh CAM, Carlsbad, CA (US); Michael Louis Losordo, San Juan Capistrano, CA (US); Jianlu Ma, Irvine, CA (US); Sanjay Shrivastava, Irvine, CA (US); Xiaoling Zhao, Irvine, CA (US)

(73) Assignee: TYCO HEALTHCARE GROUP LP, Mansfield, MA (US)

(21) Appl. No.: 13/473,840

(22) Filed: May 17, 2012

Related U.S. Application Data
(60) Provisional application No. 61/488,128, filed on May 19, 2011.

(51) Int. Cl. A61M 29/00

(52) U.S. Cl. 606/191

(57) ABSTRACT

A vascular remodeling device is provided that can comprise a proximal section, an intermediate section, and a distal section. During deployment, the proximal section can expand from a compressed delivery state to an expanded state and anchor the device in the afferent vessel of a bifurcation. The distal section can comprise at least one embolization coil that can be positioned within an aneurysm to treat the aneurysm and expand from the compressed delivery state to an expanded state upon deployment. The intermediate section can allow perfusion to efferent vessels. Before, during, and/or after the device is positioned, additional embolic material can be used to treat the aneurysm.

### CROSS-REFERENCE TO RELATED APPLICATIONS

This application claims the benefit of U.S. Provisional Application No. 61/488,128, filed May 19, 2011, the entirety of which is incorporated herein by reference.

### BACKGROUND

1. Field

The present application generally relates to vascular remodeling devices and to the manner of their positioning in vessels, and, more particularly, to remodeling devices having embolization coil distal sections and to the manner of their positioning at the junction of neurovascular bifurcations having an aneurysm and to remodeling devices having embolic protecting distal sections and to the manner of their use for clot retrieval.

2. Description of Related Art

Neurovascular or cerebral aneurysms affect about 5% of the population. Aneurysms can be located, for example, along arterial side walls (e.g., the aneurysm 10 illustrated in FIG. 1) and at arterial bifurcations (e.g., the aneurysm 20 illustrated in FIG. 2). The direction of fluid flow is generally indicated by the arrows 16, 26. The aneurysms 10, 20 each have a fundus 12, 22, a neck 14, 24, and a fundus-to-neck ratio or “neck ratio.” If the neck ratio is greater than 2 to 1 or if the neck 14, 24 is less than 4 mm, the aneurysm 10, 20 can be treated with embolization coils alone because the coils will generally constrain themselves within the aneurysm 10, 20 without dislodging into parent vessels. If the neck ratio is less than 2 to 1 or if the neck 14, 24 is greater than 4 mm, the aneurysms 10, 20 can be difficult to treat with embolization coils alone because the coils can be prone to dislodging into parent vessels, as illustrated in FIGS. 3A and 3B. Herniation, prolapse, or dislodging of coils can cause arterial occlusion, stroke, and/or death. Compared to the bifurcation illustrated in FIG. 2, the efferent vessels of the bifurcation can be at substantially different angles, have substantially different sizes, and/or be a different quantity (e.g., three or more). Compared to the bifurcation illustrated in FIG. 2, the aneurysm 20 of the bifurcation can be offset with respect to the junction (e.g., having a neck substantially open to one efferent vessel), tilted with respect to a plane created by the vessels (e.g., into or out of the page), etc. Moreover, vasculature can include more than two efferent vessels (e.g., three efferent vessels in a trifurcation). Each of these would still be accurately characterized as a “bifurcation” herein.

In order to inhibit such dislodging, tubular neck remodeling devices, for example Neuroform®, available from Boston Scientific, and Enterprise™, available from Cordis Neurovascular, may be used to keep coils or other materials within the fundus of the aneurysm and out of the vessels. Tubular remodeling devices generally consist of a braided wire or cut metallic stent or stents covering the neck of the aneurysm so that materials introduced into the fundus of the aneurysm do not dislodge out of the aneurysm. As illustrated in FIG. 4A, tubular remodeling devices 40 are generally useful for side wall aneurysms 10. As illustrated in FIGS. 4B and 4C, tubular remodeling devices 42, 44 are generally less useful for aneurysms 20 at bifurcations (e.g., the basilar tip area), for example because positioning/shaping the remodeling devices to preserve blood flow through the afferent and efferent vessels while also inhibiting dislodging of coils 28 out of the aneurysm 20 can be difficult.

### SUMMARY

In some embodiments described herein, an intraluminal vascular remodeling device or stent includes a tubular proximal portion and a distal portion. The proximal portion has an open cell design, a closed cell design, or a hybrid cell design having no reverse free-peaks for retrievability, good flexibility, and/or good wall apposition, or can be braided from a plurality of filaments. The proximal portion can include one or more tapered portions that allow the device to be retrievable. The distal portion includes at least one embolization coil. The distal portion can include a plurality of embolization coils. At least one embolization coil can comprise a platinum-tungsten alloy. Other materials are possible (e.g., shape-memory material, radiopaque material). The embolization coils can be of different types (e.g., standard helical, helical with varying diameter and/or pitch, 3D, combinations of the same, and the like). At least one of the embolization coils can be a framing coil. The embolization coils can be of varying properties (stiffness, flexibility, etc). The proximal portion is connected to the distal portion by an intermediate portion that can include a plurality of straight or elongation struts or a unit cell of the proximal portion. The delivery device for the stent includes an outer sheath (e.g., a microcatheter) containing the stent in the compressed delivery state and a plunger configured to push the stent out of the outer sheath and to release the stent mechanically, chemically, or electrolytically. The plunger can also include a guidewire lumen for aid in positioning of the delivery device at the treatment area. During deployment, the distal portion expands from the compressed delivery state to an expanded state within the fundus of the aneurysm. The proximal portion is positioned in an afferent vessel and an intermediate portion couples the proximal portion to the distal portion. The intermediate portion does not interfere with blood flow to efferent vessels. The distal portion can be configured to act as a scaffolding to prevent dislodging of objects out of the neck and/or fundus of the bifurcation aneurysm. The distal portion can be configured to allow insertion of embolic material therethrough.

For purposes of summarizing the inventions and the advantages that may be achieved over the prior art, certain objects and advantages of the inventions are described herein. Of course, it is to be understood that not necessarily all such objects or advantages need to be achieved in accordance with any particular embodiment. Thus, for example, those skilled in the art will recognize that the inventions may be embodied or carried out in a manner that achieves or optimizes one advantage or group of advantages as taught or suggested herein without necessarily achieving other objects or advantages as may be taught or suggested herein.

In accordance with some embodiments, an intraluminal device is provided that can comprise a proximal section, an intermediate section, and a distal section. The proximal section can be configured to anchor in an afferent vessel. The intermediate section can be configured to self-expand and allow perfusion to efferent vessels. Further, the distal section can comprise an embolization coil. The embolization coil can be coupled to and extend distally from the intermediate section and configured to be positioned within an aneurism. According to some embodiments, the distal section can be fabricated separately and can be then attached to the intermediate section.

According to some embodiments, the proximal section can comprise a hybrid cell design. The proximal section can also be configured to comprise a plurality of repeating unit cells. The proximal section can also be configured to comprise a plurality of woven filaments. The proximal section can also be configured to comprise a tapered portion. Further, the proximal section can also be configured to comprise a plurality of tapered portions. The proximal section can also be configured to have a length between about 5 mm and about 30 mm. Further, the proximal section can be configured to have a length between about 10 mm and about 20 mm. Furthermore, in some embodiments, the proximal section can be configured to comprise the intermediate section.

In some embodiments, the intermediate section can be configured to have a length between about 0 mm and about 6 mm. The intermediate section can be configured to comprise a plurality of struts. The intermediate section can be configured to comprise an elongation strut.

Further, some embodiments of the device can be configured such that the embolization coil of the distal section comprises a standard helical coil. The embolization coil can also comprise a helical coil with varying diameter and/or pitch. Further, the embolization coil can also be configured to comprise a 3D coil. The embolization coil can be configured to comprise platinum, a platinum-tungsten alloy, or a platinum-iridium alloy. The embolization coil can also be configured to comprise a shape memory material. The embolization coil can also be configured to comprise a radiopaque material. The embolization coil can be configured to be generally stiff. The embolization coil can be configured as a framing coil. Further, the embolization coil can be generally flexible. Furthermore, the embolization coil can be a filler coil.

In accordance with some embodiments, the distal section can comprise a plurality of embolization coils. For example, the plurality of embolization coils can be two embolization coils. However, the plurality of embolization coils can also be greater than two embolization coils.

In some embodiments, the embolization coil can be a 3-D coil. For example, in embodiments wherein the distal section comprises a plurality of embolization coils, at least one embolization coil of the plurality of embolization coils can be a 3D coil. Further, in some embodiments, at least one embolization coil of the plurality of embolization coils can be a standard helical coil. Further, at least one embolization coil of the plurality of embolization coils can be a helical coil with varying diameter and/or pitch. In some embodiments, combinations of types of embolization coils can be used in the plurality of embolization coils.

In some embodiments, the proximal section and the distal section can be formed from the same or different materials. For example, the proximal section can comprise a first material and the distal section can comprise a second material that is different from the first material. The first material can comprise a shape-memory material. For example, the first material can comprise Nitinol. Further, the first material can comprise CoCr alloy. Additionally, the first material can comprise a radiopaque material. The second material can comprise platinum, a platinum-iridium alloy, or a platinum-tungsten alloy. The second material can also comprise a radiopaque material.

In accordance with some embodiments, the device can comprise an insulating material. For example, the first material can be insulated from the second material. Further, the device can be configured to comprise an insulating coating over an intersection between the first material and the second material.

In accordance with yet other embodiments, a method is provided for treating an aneurysm near a junction of a bifurcation having an afferent vessel and efferent vessels. The aneurysm can define a neck and a fundus. In some embodiments, the method can comprise advancing a first catheter proximate to the junction of the bifurcation. The catheter can at least partially contain a device in a compressed state. The device can include a proximal section, and intermediate section, and a distal section. The proximal section can be configured to anchor in an afferent vessel. The intermediate section can be configured to self-expand and allow perfusion to efferent vessels. Further, the distal section can comprise an embolization coil. The embolization coil can be coupled to and extend distally from the intermediate section and configured to be positioned within an aneurism. In addition, the device can be configured to comprise any of the features discussed above and further herein.

The method can further comprise deploying the device from at least partially inside the first catheter to outside the first catheter at the junction of the bifurcation. Further, during deployment, the distal section can self-expand within the fundus of the aneurysm. Additionally, the intermediate section can self-expand and allow perfusion to the efferent vessels. Furthermore, the proximal section can self-expand to anchor in an expanded state to the walls of the afferent vessel.

In accordance with some embodiments, the method can be implemented such that deploying the device comprises initially deploying the device, retrieving at least a section of the device at least partially back into the first catheter, and redeploying the device. Further, the method can be implemented such that deploying the device comprises releasing the device from the first catheter. The method can also be implemented such that releasing the device from the first catheter comprises mechanical detachment. The method can also be implemented such that releasing the device from the first catheter comprises electrolytic detachment. The method can also be implemented such that releasing the device from the first catheter comprises chemical detachment.

The method can also be configured to further comprise inserting additional embolic material into the aneurysm. Further, the method can be implemented such that inserting the additional embolic material comprises deploying the additional embolic material from the first catheter. The method can also be implemented such that inserting the additional embolic material comprises deploying the additional embolic material from a second catheter. In some embodiments, the method can be implemented such that inserting the additional embolic material is before deploying the device. Further, the method can be implemented such that inserting the additional embolic material is after deploying the device. Additionally, the method can be implemented such that inserting the additional embolic material is during deploying the device. The method can be implemented such that inserting the additional embolic material comprises inserting embolic coils. The method can also be implemented such that inserting the additional embolic material comprises inserting embolic fluid. Further, the method can be implemented for situations in which the aneurysm comprises a basilar tip aneurysm.

According to yet other embodiments disclosed herein, a system for treating aneurysms is provided that can comprise a plurality of intraluminal devices. For example, the system can comprise first and second intraluminal devices. The first and second intraluminal devices can each comprise a proximal section, and intermediate section, and the distal section. The proximal section can be configured to anchor in an afferent vessel. The intermediate section can be configured to self-expand and allow perfusion to efferent vessels. The distal section can comprise an embolization coil. In accordance with some embodiments, the distal section of the first device can have at least one property that is different from a corresponding property of the second device. For example, as discussed below, in some embodiments, the distal section of the first device can have a different thickness, cross-section or profile, flexibility, coil packing density, etc., than the distal section of the second device.

In some embodiments, the system can comprise a catalogue from which one or more of the plurality of intraluminal devices can be selected. At least one intraluminal device of the system can be configured in accordance with any of the features noted above and herein.

For example, the first intraluminal device of the system can comprise a distal section having a first kind of embolization coil and the second intraluminal device of the system can comprise a distal section comprising a second kind of embolization coil. In some embodiments, the first kind of embolization coil can be a standard helical coil. Further, the first kind of embolization coil can be a helical coil with varying diameter and/or pitch. The first kind of embolization coil can be a 3D coil. Furthermore, the first kind of embolization coil can be a 3D helical coil.

In some embodiments, the second kind of embolization coil can be a standard helical coil. The second kind of embolization coil can be a helical coil with varying diameter and/or pitch. The second kind of embolization coil can be a 3D coil. Further, second kind of embolization coil can be a 3D helical coil.

Optionally, the first intraluminal device of the system can comprise a distal section having an embolization coil of one length and the second intraluminal device of the system can comprise a distal section having an embolization coil of a second length. For example, the first length can be between about 0.04 inches and about 1 inch (approx. between about 1 mm and about 25 mm) and the second length can be between about 1 inch and about 15 inches (approx. between about 25 mm and about 380 mm).

In some embodiments, the first intraluminal device can comprise a first embolization coil and the second intraluminal device can comprise a second embolization coil. The first and second embolization coils can have different characteristics, such as different flexibilities, different packing densities, different cross-sections or profiles, different thicknesses, etc. For example, the first embolization coil can have a first flexibility and the second embolization coil can have a second flexibility that is greater than the first flexibility. Further, in some embodiments, the first embolization coil can have a first packing density and the second embolization coil can have a second packing density that is greater than the first packing density. Additionally, in some embodiments, the first embolization coil can have a first cross-section and the second embolization coil can have a second cross-section that is greater than the first cross-section. In some embodiments, the first embolization coil can have a first length and the second embolization coil can have a second length that is greater than the first length.

Furthermore, the first embolization coil can comprise a first material and the second embolization coil can comprise a second material different than the first material. For example, the first material can comprise platinum, platinum-iridium alloy, or platinum-tungsten alloy. Further, the second material can comprise a shape-memory material.

Additionally, the first embolization coil can be configured to comprise a first base shape than the second embolization coil can be configured to comprise a second base shape different than the first base shape. For example, the first base shape can be configured to comprise a wire or filament. Further, the second base shape can be configured to comprise a coil.

In accordance with some embodiments, the first embolization coil can comprise a first pitch and the second embolization coil can comprise a second pitch different than the first pitch. In some embodiments, the distal section of the first intraluminal device can comprise a first number of embolization coils and the distal section of the second intraluminal device can comprise a second number of embolization coils different than the first number of embolization coils.

In accordance with some embodiments, the first intraluminal device can comprise the first embolization coil and the second intraluminal device can comprise a second embolization coil. In some embodiments, the system can be configured such that the embolization coil of the distal section of the second device has a different property than the embolization coil of the distal section of the first device. For example, as discussed below, in some embodiments, the distal section of the first device can have a different thickness, cross-section or profile, flexibility, coil packing density, etc., than the distal section of the second device.

In accordance with some embodiments, the property can comprise thickness. The property can also comprise cross-section or profile. The property can also comprise diameter. The property can also comprise pitch. Further, the property can also comprise shape. The property can also comprise type. The property can also comprise base shape. The property can also comprise material(s). The property can also comprise flexibility. One or more of the above-noted properties, various other types of properties, or a combination of such properties can be used in some embodiments.

According to some embodiments, a method is provided for manufacturing an intraluminal device. The method can comprise assembling a proximal section configured to anchor in an afferent vessel. The intermediate section can be configured to self-expand and allow perfusion to efferent vessels. Further, a distal section can be configured to comprise an embolization coil.

In some embodiments, the method can further comprise forming the proximal section. Further, the method can be implemented such that forming the proximal section comprises cutting the proximal section from a tube. The method can also be implemented such that forming the proximal section further comprises rolling sides of the sheet to faun a tubular profile. The method can also be implemented such that forming the proximal section comprises cutting the proximal section from a tube.

In accordance with some embodiments, the method can further comprise forming the intermediate section. The method can be implemented such that forming the intermediate section comprises shape setting the intermediate section so that at least a portion of the intermediate comprises a diameter greater than the diameter of the proximal section. Further, the method can be implemented such that forming the intermediate section comprises forming at least one strut. The method can also be implemented such that forming the intermediate section comprises integrally forming the intermediate section and the proximal section.

In some embodiments, the method can be implemented such that integrally forming the intermediate section and the proximal section comprises cutting the intermediate section and the proximal section from a tube. Further, integrally forming the intermediate section and the proximal section can further comprise rolling sides of the sheet to form a tubular profile. Additionally, integrally forming the intermediate section and the proximal section can further comprise cutting the intermediate section and the proximal section from a sheet. Moreover, the method can be implemented such that forming the intermediate section is separate from formation of the proximal section, and the method can further comprise coupling the proximal section and the intermediate section.

Some embodiments, the method can further comprise forming the distal section. The method can be implemented such that forming the distal section comprises integrally forming the intermediate section and the distal section. Method can also be implemented such that forming the distal section is separate from formation of the intermediate section, and wherein the method further comprises coupling the distal section and the intermediate section.

All of these embodiments are intended to be within the scope of the inventions herein disclosed. These and other embodiments will become readily apparent to those skilled in the art from the following detailed description having reference to the attached figures, the inventions not being limited to any particular disclosed embodiment(s).

### BRIEF DESCRIPTION OF THE DRAWINGS

These and other features, aspects, and advantages of the present disclosure are described with reference to the drawings of certain embodiments, which are intended to illustrate certain embodiments and not to limit the inventions.

FIG. 1 illustrates an example embodiment of a side wall aneurysm.

FIG. 2 illustrates an example embodiment of a bifurcation having an aneurysm.

FIG. 3A illustrates an example embodiment of a side wall aneurysm with herniating embolization coils.

FIG. 3B illustrates an example embodiment of a bifurcation having an aneurysm with herniating embolization coils.

FIG. 4A illustrates an example embodiment of a side wall aneurysm treated with embolization coils and a tubular remodeling device.

FIGS. 4B and 4C illustrates example embodiments of a bifurcation having an aneurysm treated with embolization coils and tubular remodeling devices.

FIG. 5 is a side elevational view of an example embodiment of a vascular remodeling device.

FIGS. 6A-6D illustrate example embodiments of embolization coils.

FIGS. 7A and 7B illustrate an example embodiment of a method for treating an aneurysm using the device of FIG. 5.

FIG. 8A illustrates an example embodiment of a cut patterns in a hypotube for forming a portion of the device of FIG. 5.

FIG. 8B illustrates the cut pattern of FIG. 9A rotated 90°.

FIG. 9 illustrates a side elevational view of another example embodiment of a vascular remodeling device.

FIG. 10 illustrates an example embodiment of an aneurysm treated using the device of FIG. 9.

FIG. 11 illustrates a side elevational view of another example embodiment of a vascular remodeling device.

FIG. 12 illustrates an example embodiment of an aneurysm treated using the device of FIG. 11.

FIG. 13 illustrates an example embodiment of a cut pattern in a sheet or a hypotube for forming a portion of the device of FIG. 9A.

FIGS. 14A-14J illustrate example embodiments of proximal sections of vascular remodeling devices.

FIGS. 15A and 15B illustrate example embodiments of intermediate sections of vascular remodeling devices.

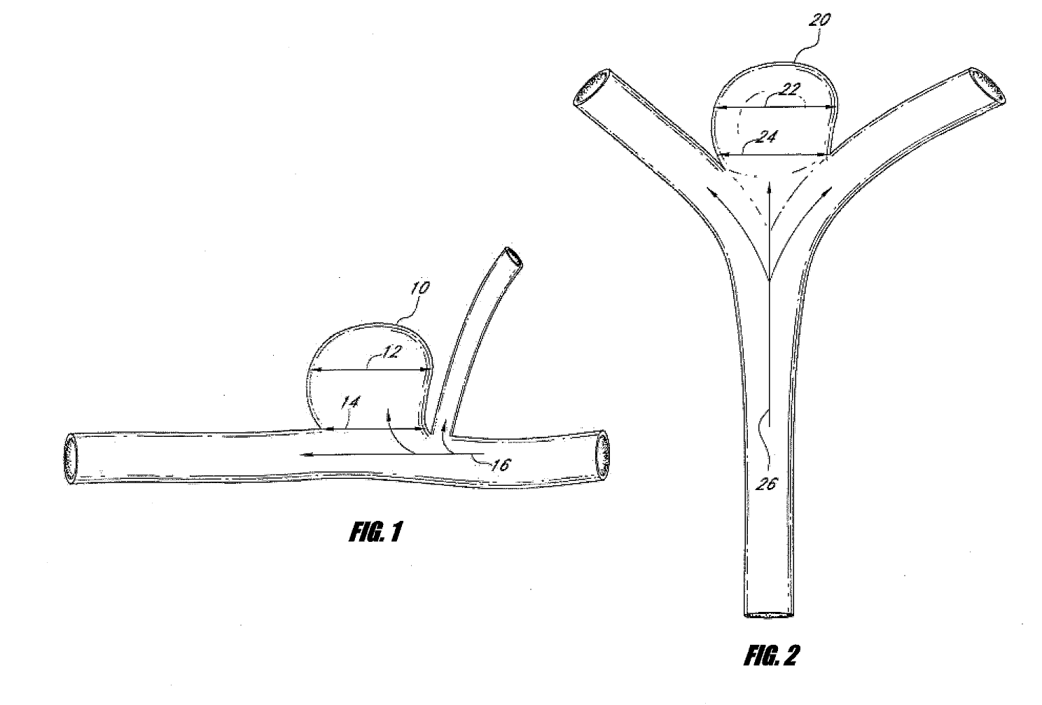

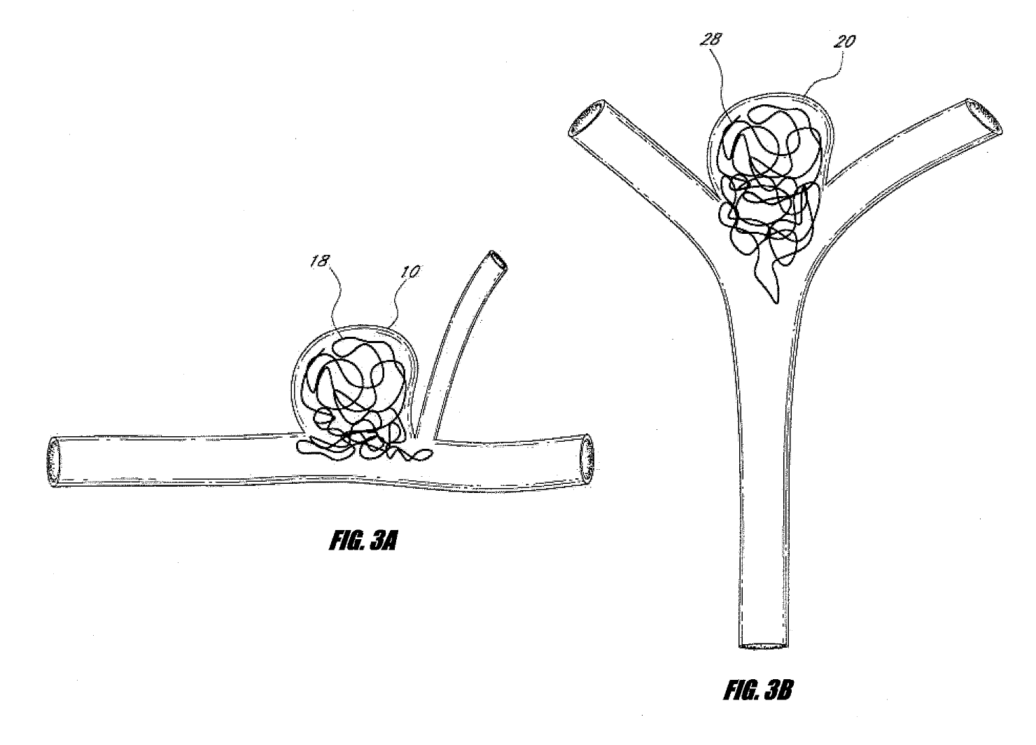

### DETAILED DESCRIPTION

Although certain embodiments and examples are described below, those of skill in the art will appreciate that the inventions extend beyond the specifically disclosed embodiments and/or uses and obvious modifications and equivalents thereof. Thus, it is intended that the scope of the inventions herein disclosed should not be limited by any particular embodiments described below.

According to some embodiments, a vascular remodeling intraluminal device can be provided that comprises an anchor section and an integrated coil distal section. The device can comprise a proximal section (or “bottom section” or “main body” or “stem” or “tubular portion” or “anchoring section”), an intermediate section (or “middle section” or “open portion” or “flow section”), and a distal section (or “top section” or “distal portion” or “coil portion” or “integrated coil section” or “treatment section”). Further, the intermediate section can comprise at least one strut that couples the proximal section to the distal section.

The intraluminal device can be delivered via a catheter (e.g., microcatheter) into a bifurcation to treat an aneurysm with minimal interruption of blood flow in afferent and efferent vessels. In some embodiments, the device can be retrieved and/or repositioned.

The strut can be straight, curved, or otherwise shaped, such as having design features like the proximal section with the same or a different cell size. The strut can also bias the distal section away from the proximal section (e.g., into an aneurysm).

In some embodiments, the strut, in conjunction with the proximal section, can bear the weight of the distal section, allowing the distal section to maintain its position within the fundus of an aneurysm. The strut can have a variety of cross-sectional shapes. For example, in some embodiments, the strut has a substantially rectangular or flat cross section (e.g., embodiments in which the strut comprises a ribbon or uncut portion of a metallic tube or sheet). Further, in some embodiments, the strut can have a substantially rounded (e.g., circular, elliptical, ovoid) cross section (e.g., embodiments in which the strut comprises a round filament).

In some embodiments, the at least one strut can comprise a plurality of struts. For example, in some embodiments, the plurality of struts comprises two struts. In some embodiments, the plurality of struts comprises greater than two struts. In some embodiments, the plurality of struts can comprise between about two struts and about twelve struts (e.g., between about three struts and about eight struts, three struts, four struts, five struts, six struts, seven struts, or eight struts). Other numbers of struts are also possible.

The number of struts can be selected, for example, based on the expected weight of the distal section (e.g., the weight of the embolization coils). For example, as coil weight increases, the number of struts can increase. For another example, the number of struts can be selected based on the number of the embolization coils in the distal section. Each embolization coil in the distal section, for example, can correspond to an individual strut. Ends of each embolization coil in the distal section, for example, can correspond to an individual strut.

In certain embodiments, the struts can be equally spaced and/or oriented on opposite sides of the intraluminal device (e.g., two struts 180° apart along the circumference of the device, three struts 120° apart along the circumference of the device, four struts 90° apart along the circumference of the device, etc.). When the device is placed at a bifurcation, the intermediate section can self-expand and allows perfusion of blood to efferent vessels because the strut does not block fluid flow.

In certain embodiments, the at least one strut can be integrally fabricated with the proximal section (e.g., by being cut from the same tube) or the distal section (e.g., by being an extension of a coil). In certain embodiments, the at least one strut can be made from a different piece and is attached (e.g., welded, glued, adhered, mechanically crimped, mechanically swaged, braided, physical vapor deposited, chemical vapor deposited, etc.) to each of the proximal section and the distal section.

In some embodiments, the at least one strut can be formed from one or more materials. For example, the at least one strut can be configured such that the proximal section is formed from a different material than the distal section. Further, in some embodiments, the at least one strut can comprise multiple sections comprising different metals.

In some embodiments, the at least one strut can comprise a biocompatible metal and/or biocompatible polymer. In some embodiments, the at least one strut comprises a radiopaque material (e.g., in the form of a radiopaque core, cladding, coating, small coiled wire, marker band, etc.), which can act as radiopaque markers for improved visibility of the device during a procedure and/or following optional implantation.

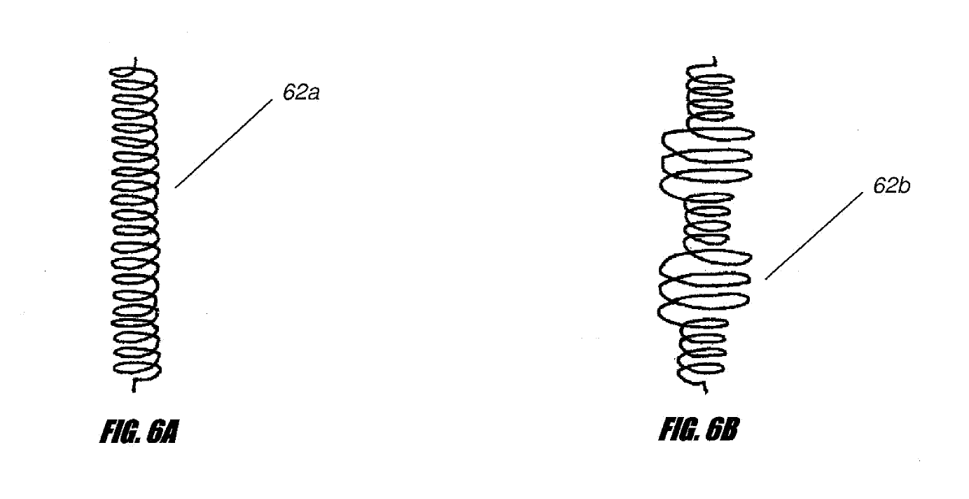

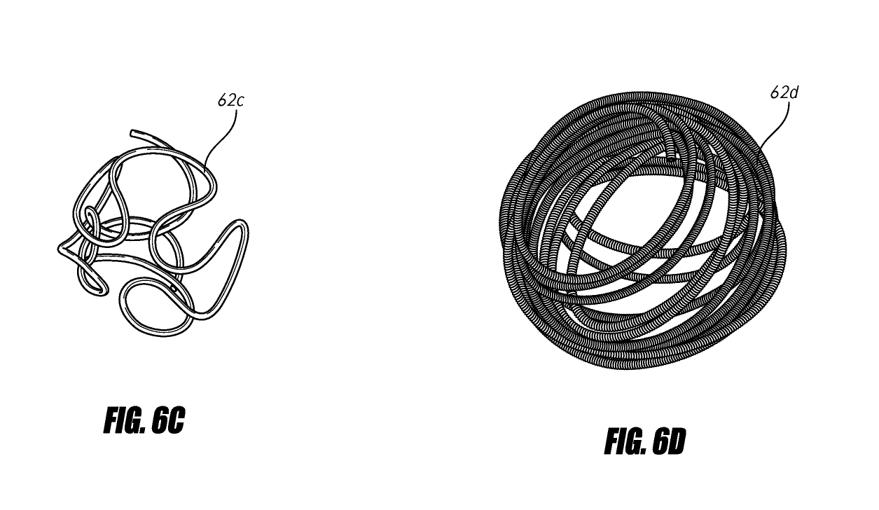

In some embodiments, a combination of different types of coils 162 can be used in the distal section 156 of a single intraluminal device 150. For example, with reference to the coils described in FIGS. 6A-6D, the device 150 can comprise a distal section 156 comprising 3D coils 62c and standard helical coils 62a. For another example, a device 150 can comprise 3D helical coils 62d and helical coils of varying diameter and/or pitch 62b. For another example, the intraluminal device 150 can comprise 3D coils 62c and helical coils of varying diameter and/or pitch 62b. For another example, the intraluminal device 150 can comprise 3D helical coils 62d and standard helical coils 62a. For another example, the intraluminal device 150 can comprise standard helical coils 62a and helical coils of varying diameter and/or pitch 62b. For another example, the intraluminal device 150 can comprise 3D helical coils 62d and 3D coils 62c. For another example, the intraluminal device 150 can comprise 3D coils 62c, 3D helical coils 62d, and helical coils of varying diameter and/or pitch 62b. For another example, the intraluminal device 150 can comprise 3D coils 62c, standard helical coils 62a, and helical coils of varying diameter and/or pitch 62b. Other combinations of coils are also possible.

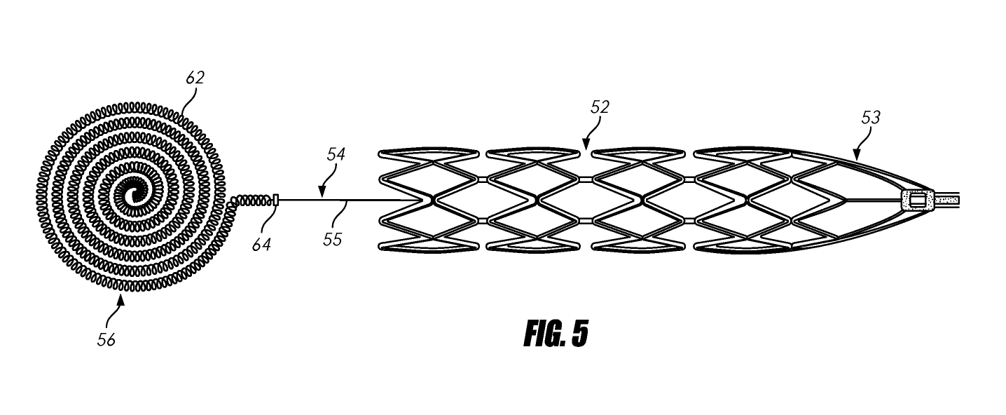

Referring now to the embodiments illustrated in the figures, FIG. 5 illustrates an example embodiment of a vascular remodeling intraluminal device 50 comprising an anchor section 52 and an integrated coil distal section 56. The intraluminal device 50 can be more compliant than the vasculature in which it is deployed such that it can be somewhat misshapen after being deployed, and that certain shapes described herein are when the device 50 is an expanded state with no restriction. The device 50 comprises a proximal section 52 (or “bottom section” or “main body” or “stem” or “tubular portion” or “anchoring section”), an intermediate section 54 (or “middle section” or “open portion” or “flow section”), and a distal section 56 (or “top section” or “distal portion” or “coil portion” or “integrated coil section” or “treatment section”). The device 50 can be delivered via a catheter (e.g., microcatheter) into a bifurcation to treat an aneurysm with minimal interruption of blood flow in afferent and efferent vessels. In some embodiments, the device 50 can be retrieved and/or repositioned.

The intermediate section 54 comprises at least one strut 55. The strut 55 can be straight, curved, or otherwise shaped, such as having design features like the proximal section 52 with the same or a different cell size. The strut 55 couples the proximal section 52 to the distal section 56. The strut 55 can also bias the distal section 56 away from the proximal section 52 (e.g., into an aneurysm). In some embodiments, the strut 55, in conjunction with the proximal section 52, bears the weight of the distal section 56, allowing the distal section 56 to maintain its position within the fundus of an aneurysm.

In some embodiments, the strut 55 can have a substantially rectangular or flat cross section (e.g., embodiments in which the strut 55 comprises a ribbon or uncut portion of a metallic tube or sheet). In some embodiments, the strut 55 can have a substantially round (e.g., circular, elliptical, ovoid) cross section (e.g., embodiments in which the strut 55 comprises a round filament).

In some embodiments, the at least one strut 55 can comprise a plurality of struts. In some embodiments, the plurality of comprises two struts. In some embodiments, the plurality of struts comprises greater than two struts. In some embodiments, the plurality of struts comprises between about two struts and about twelve struts (e.g., between about three struts and about eight struts, three struts, four struts, five struts, six struts, seven struts, or eight struts). Other numbers of struts are also possible. The number of struts can be selected, for example, based on the expected weight of the distal section (e.g., the weight of the embolization coils). For example, as coil weight increases, the number of struts can increase. For another example, the number of struts can be selected based on the number of the embolization coils 62 in the distal section 56. Each embolization coil 62 in the distal section 56, for example, can correspond to an individual strut. Ends of each embolization coil 62 in the distal section, for example, can correspond to an individual strut. In certain embodiments, the struts can be equally spaced and/or oriented on opposite sides of the device 50 (e.g., two struts 180° apart along the circumference of the device 50, three struts 120° apart along the circumference of the device 50, four struts 90° apart along the circumference of the device 50, etc.). When the device 50 is placed at a bifurcation, the intermediate section 54 can self-expand and allows perfusion of blood to efferent vessels because the strut 55 does not block fluid flow.

In certain embodiments, the at least one strut 55 is integrally fabricated with the proximal section 52 (e.g., by being cut from the same tube) or the distal section 56 (e.g., by being an extension of a coil 62). In certain embodiments, the at least one strut 55 is made from a different piece and is attached (e.g., welded, glued, adhered, mechanically crimped, mechanically swaged, braided, physical vapor deposited, chemical vapor deposited, etc.) to each of the proximal section 52 and the distal section 56. A separately formed strut can allow the at least one strut 55 to be a different material from the proximal section 52 and the distal section 56, although flat pieces of metal can also comprise multiple sections comprising different metals. In some embodiments, the at least one strut 55 can comprise a biocompatible metal and/or biocompatible polymer. In some embodiments, the at least one strut 55 comprises a radiopaque material (e.g., in the form of a radiopaque core, cladding, coating, small coiled wire, marker band, etc.), which can act as radiopaque markers for improved visibility of the device 50 during a procedure and/or following optional implantation.

In some embodiments, the device 50 comprises an anchor or proximal section 52 that is flexible and yet has enough radial force to anchor or maintain the position of the device 50 at a bifurcation after deployment. For example, the proximal section 52 can be configured to inhibit or prevent longitudinal migration of the device 50. In some embodiments, the proximal section 52 has enough radial force to bear the weight of the intermediate section 54 and the distal section 56.

In certain embodiments, the proximal section 52 has a first diameter and the distal section 56 has a second diameter greater than the first diameter (e.g., due to expansion of the integrated coils 62 within the aneurysm), which can cause the strut 55 to be angled or curved outwards from the longitudinal axis defined by the proximal section 52.

In certain embodiments, the proximal section 52 has a round (e.g., circular, elliptical, or ovoid) cross section. In some embodiments, the proximal section 52 includes filaments having a substantially rectangular or flat cross section (e.g., embodiments in which the proximal section 52 comprises ribbons or uncut portions of a metallic tube or sheet). In some embodiments, the proximal section 52 includes filaments having a substantially round (e.g., circular, elliptical, ovoid) cross section (e.g., embodiments in which the proximal section 52 comprises round filaments).

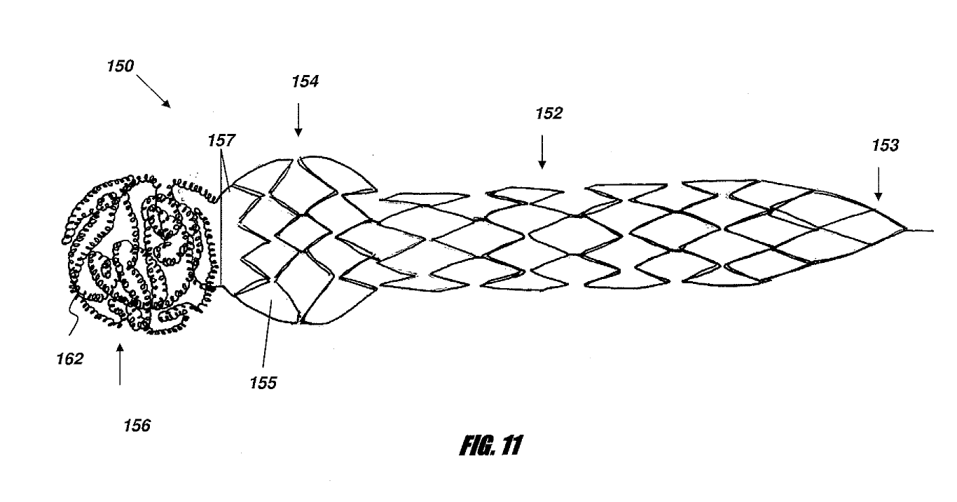

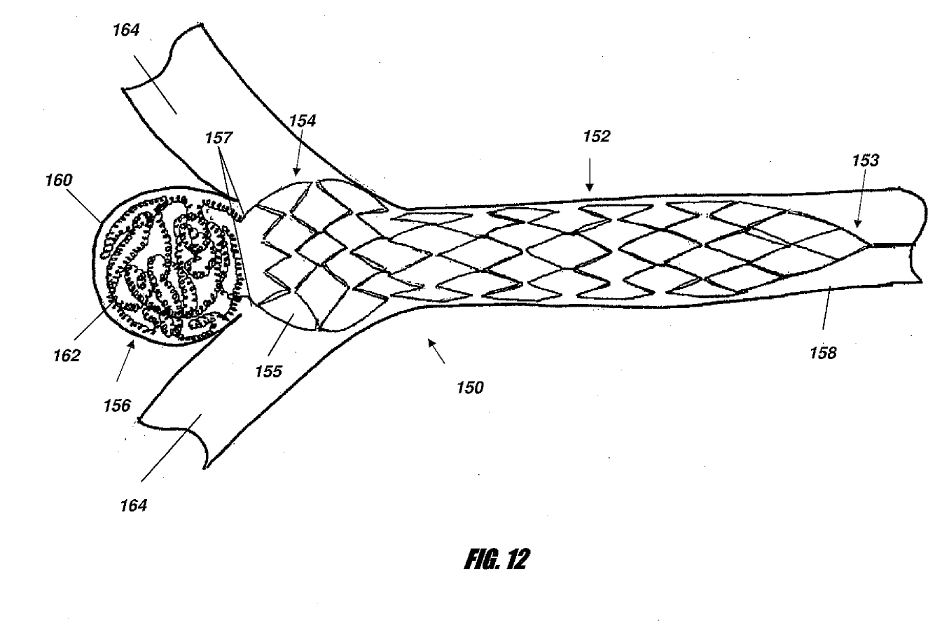

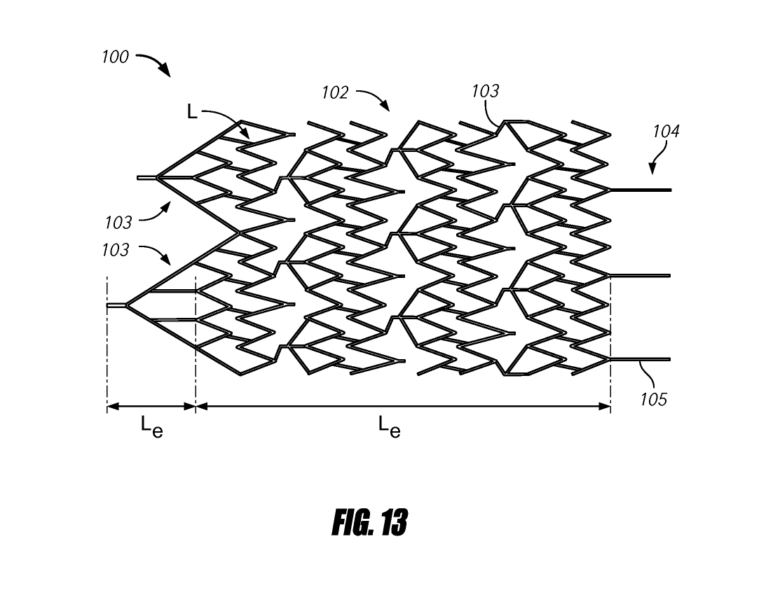

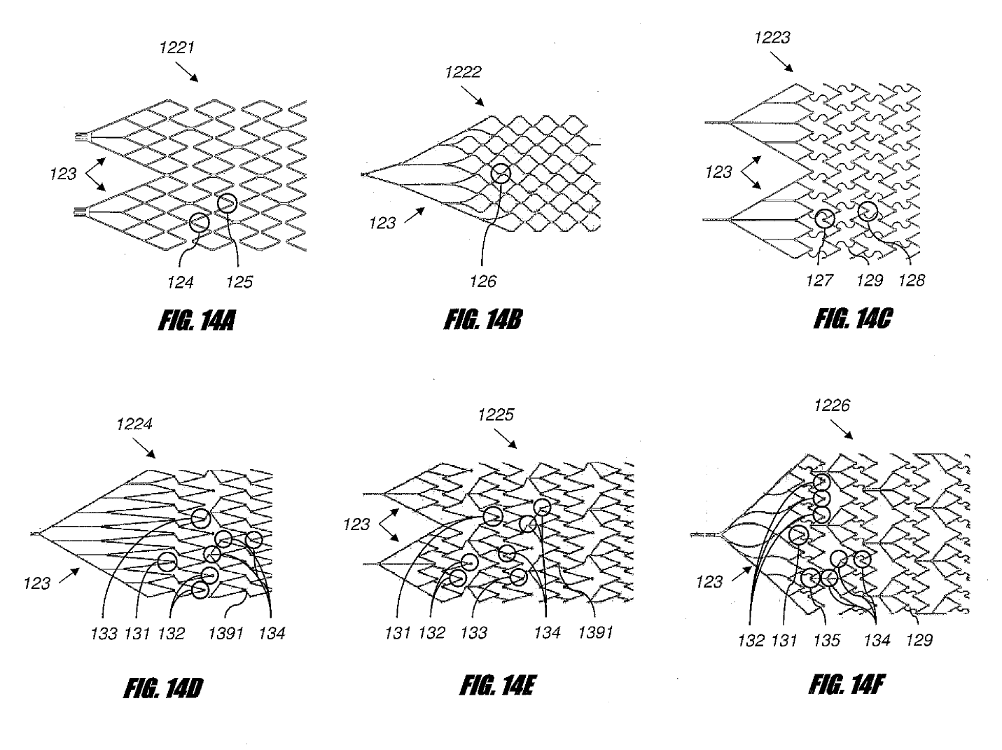

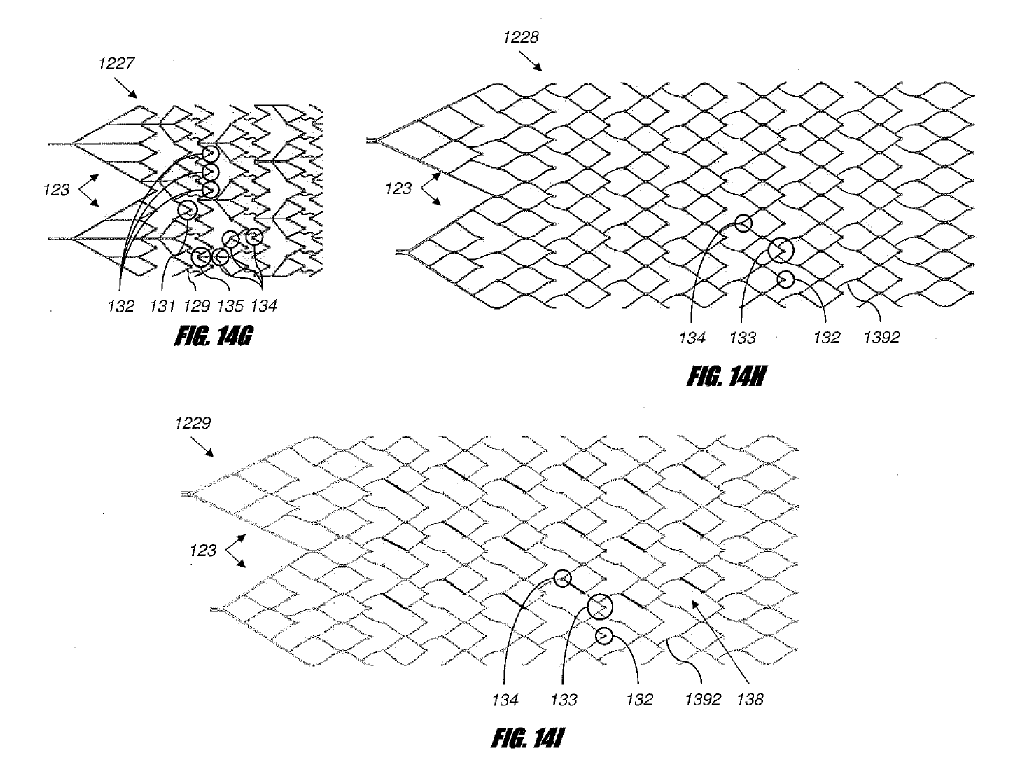

In some embodiments, the proximal section 52 comprises a plurality of z-shaped segments coupled by struts (e.g., as illustrated in FIG. 5). Other patterns of the proximal section 52 are also possible, for example as described with respect to FIGS. 14A-14J. When the device 50 is placed at a bifurcation, the proximal section 52 provides anchoring of the device 50 in the afferent vessel. The proximal section 52 can also facilitate delivery, positioning, retrieval, and/or repositioning of the device 50.

In the embodiment illustrated in FIG. 5, the proximal end of the proximal section 52 can comprise at least two tapered portions 53. The tapered portions 53 can allow the device 50 or portions thereof (e.g., the proximal section 52) to be retrieved back into a catheter. For example, if the device 50 is being pulled into a catheter, the tapered portions 53 can radially compress the proximal section 52. One tapered portion 53 or other numbers of tapered portion 53 are also possible.

In some embodiments, the embolization coil can be coupled to and extend distally from the intermediate section and configured to be positioned within an aneurism. For example, the distal section can comprise embolization coils that can be placed within the fundus of an aneurysm to treat the aneurysm.

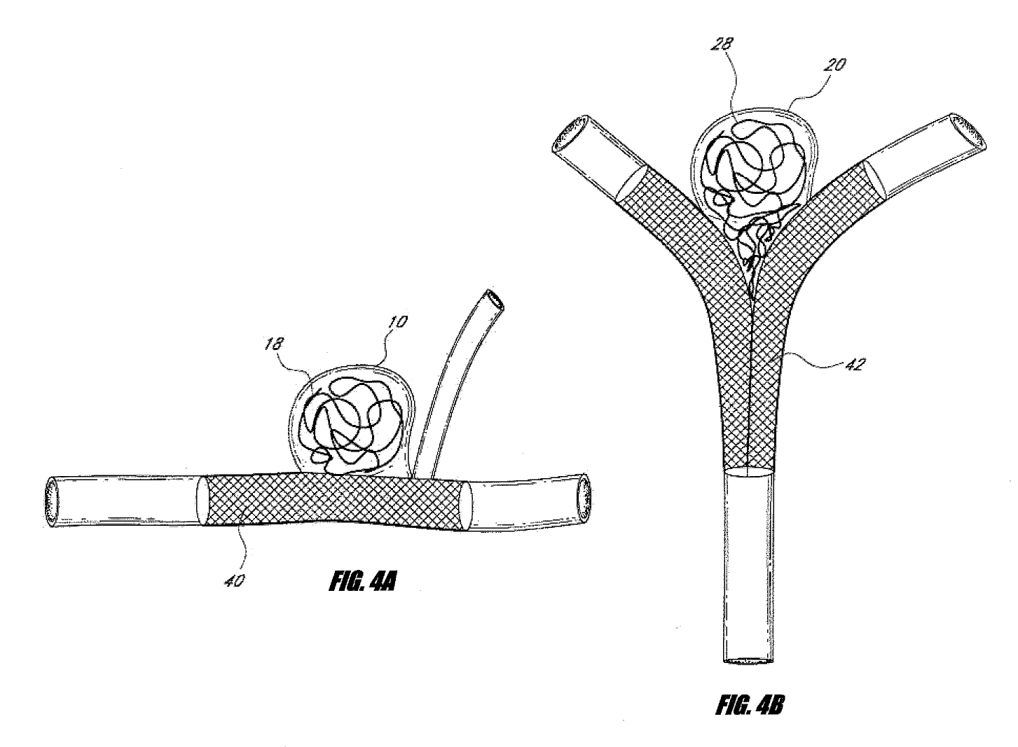

FIGS. 6A-6D illustrate example embodiments of embolization coils 62 that can be incorporated into the distal section 56. Embolization coils generally comprise a thin strand of material that can be adapted to assume a variety of shapes when not confined. In some embodiments, the distal section 56 can comprise standard helical embolization coils 62a as shown in FIG. 6A. In some embodiments, the distal section 56 comprises helical embolization coils 62b with varying diameter and/or pitch as shown in FIG. 6B. In some embodiments, the distal section 56 comprises three-dimensional (3D) embolization coils 62 (e.g., ev3 Axium® Coils). FIGS. 6C and 6D depict embodiments of 3D embolization coils 62c. The embolization coil 62c of FIG. 6C comprises a base shape of a wire or filament that has been heat set to take on a complex configuration. In some embodiments, the embolization coil 62c of FIG. 6C comprises a different base shape (e.g., coil). 3D embolization coils can also comprise helical coils 62d that have been heat set to take on a more complex 3-dimensional shape, for example like the embolization coils 62d shown in FIG. 3D. Other coil configurations are also possible (e.g., a braided configuration, a twisted configuration, etc.). In some embodiments, the distal section 56 comprises one embolization coil 62. In some embodiments, the distal section 56 comprises two embolization coils 62. Other numbers of embolization coils 62 are also possible (three, four, five, six, etc.). In some embodiments, the embolization coils 62 comprises a coil diameter of between about 0.04 inches and about 1 inch (approx. between about 1 mm and about 25 mm). In some embodiments, the embolization coils 62 comprises a coil length of between about 0.4 inches and about 20 inches (approx. between about 10 mm and about 510 mm).

A system of intraluminal devices can be configured such that different intraluminal devices comprise distal sections with different properties. In accordance with some embodiments, the distal section of a first intraluminal device can be selected to have at least one property that is different from a corresponding property of a second intraluminal device. For example, as discussed herein, in some embodiments, the distal section of the first device can have a different thickness, cross-section or profile, flexibility, coil packing density, etc., than the distal section of the second device.

For example, the distal sections can be different in that the coils of the distal sections have different thicknesses, cross-sections or profiles, lengths, packing densities, stiffnesses, pitches, shapes, types, materials, base shapes, etc and combinations thereof. Different shapes, sizes, and/or other properties of the coils 62 can allow for selection of a device from a system of devices that comprises a distal section that is appropriate for the particular aneurysm to be treated (e.g., based on size, shape, etc.). For instance, a physician can be able to select a device having a distal section that is most appropriate for the patient and/or vasculature to be treated (e.g., by browsing through a catalogue, by selecting from a kit, etc.).

For example, when the aneurysm has a narrow neck, an intraluminal device 50 comprising a distal section 56 comprising a thin (e.g., coil diameter of between about 0.04 inches and about 0.5 inches (approx. between about 1 mm and about 13 mm)), flexible coil 62 can be selected. For another example, when the aneurysm has a wide neck, a device 50 comprising a distal section 56 comprising a stiff, dense coil 62 can be selected. For yet another example, when the aneurysm is small, a device 50 comprising a distal section 56 comprising a short (e.g., coil length of between about 0.4 inches and about 1 inch (approx. between about 10 mm and about 25 mm)) and/or compact (e.g., coil diameter of between about 0.04 inches and about 0.5 inches (approx. between about 10 mm and about 13 mm)) coil 62 can be selected. For still yet another example, when the aneurysm is large, a device 50 comprising a distal section 56 comprising a long (e.g., coil length of between about 1 inch and about 15 inches (approx. between about 25 mm and about 380 mm)) and/or voluminous coil (e.g., coil diameter of between about 0.5 inches and about 1 inches (approx. between about 13 mm and about 25 mm)), coil 62 can be selected.

In some embodiments in which the intraluminal device 50 comprises a distal section 56 comprising a plurality of embolization coils 62, the coils 62 in the distal section 56 can have different properties (e.g., thickness, cross-section or profile, length, coil packing density, pitch, shape, type, materials, base shape etc.). Coils with differing properties can allow for selection of a device 50 comprising a distal section with properties appropriate for filling a particular aneurysm (e.g., based on size, shape, etc.). For example, when the device 50 is being used to treat an aneurysm with a wide neck, the device 50 can be configured with a distal section comprising a stiff, dense coil (e.g., to frame the neck of the aneurysm, to keep objects from protruding from the neck of the aneurysm) in combination with a thin (e.g., coil diameter of between about 0.04 inches and 1 inches (approx. between about 1 mm and about 25 mm)), flexible coil 62 (e.g., to fill the aneurysm).

In some embodiments, a combination of different types of coils 62 can be used in the distal section 56 of a single device 50. For example, a device 50 can comprise a distal section 56 comprising 3D coils 62c and standard helical coils 62a. For another example, a device 50 can comprise 3D helical coils 62d and helical coils of varying diameter and/or pitch 62b. For another example, a device 50 can comprise 3D coils 62c and helical coils of varying diameter and/or pitch 62b. For another example, a device 50 can comprise 3D helical coils 62d and standard helical coils 62a. For another example, a device 50 can comprise standard helical coils 62a and helical coils of varying diameter and/or pitch 62b. For another example, a device 50 can comprise 3D helical coils 62d and 3D coils 62c. For another example, a device 50 can comprise 3D coils 62c, 3D helical coils 62d, and helical coils of varying diameter and/or pitch 62b. For another example, a device 50 can comprise 3D coils 62c, standard helical coils 62a, and helical coils of varying diameter and/or pitch 62b. Other combinations of coils are also possible.

In some embodiments, the distal section 56 comprises embolization coils 62 that are arranged in a configuration that can provide a frame or basket to inhibit objects (e.g., thrombi, coils, embolization fluid, etc.) from protruding from the aneurysm into the junction or confluence of the bifurcation. For example, in some embodiments, the distal section 56 comprises helical framing embolization coils 62 that can aid in inhibiting objects (e.g., thrombi, coils, embolization fluid, etc.) from protruding from the aneurysm. For another example, in some embodiments, the distal section 56 comprises 3D framing embolization coils 62 that can aid in inhibiting objects (e.g., thrombi, coils, embolization fluid, etc.) from protruding from the aneurysm.

In some embodiments, the intraluminal device 50 comprises a metallic material (e.g., platinum, tungsten, tantalum, palladium, gold, titanium, silver, etc.). In some embodiments, the device 50 comprises a metal alloy (e.g., platinum alloy (e.g., platinum-tungsten, platinum-iridium), tungsten alloy, stainless steel, tantalum alloy, etc.). In some embodiments, the device 50 comprises a platinum-tungsten alloy (e.g., T10 PtW). In some embodiments, the device 50 comprises a self-expanding, super elastic, and/or a shape-memory material (e.g., comprising Nitinol, CoCr alloy, shape memory polymers (e.g., polyglycolic acid, polylactic acid), etc.), thereby causing the device 50 to be self-expanding under certain conditions (e.g., not restrained by a catheter). In some embodiments, the device 50 comprises a bioabsorbable polymer (e.g., polylactic acid, polyglycolic acid, poly(lactic-co-glycolic acid), poly-epsilon-caprolactone, and/or naturally derived bioabsorbable polymers, etc.), thereby causing the device 50 to bioabsorb over time at a rate dependent on the composition of bioabsorbable polymer(s).

In some embodiments, the proximal section 52, the intermediate section 54, and/or the distal section 56 comprises different materials. For example, the distal section 56 can comprise platinum-tungsten alloy while the proximal section 52 and the intermediate section 54 comprise Nitinol. For another example, the distal section 56 can comprise polymer material while the proximal section 52 and the intermediate section 54 comprise metallic material, different polymer material, etc. For yet another example, the distal section 56 can comprise metallic material while the proximal section 52 and the intermediate section 54 comprise different metallic materials, polymer material, etc. Other combinations of the materials described herein and other materials within a single device 50 are also possible.

The intraluminal device 50 can assume a low profile compressed state (e.g., confined within a catheter) for delivery. Upon deployment from the catheter, the device 50 expands (e.g., self-expands) from the compressed state to an expanded state. The distal section 56 comprises coils that can have a compressed or substantially linear configuration when inside the catheter and a different expanded configuration when deployed.

In some embodiments, the device 50 comprises a radiopaque material such as platinum, platinum-iridium, and/or tantalum (e.g., being at least partially formed from the radiopaque material (e.g., having a radiopaque layer, consisting of a radiopaque material), including radiopaque markers). For example, the strut 55 can comprise a radiopaque marker. For another example, certain segments of the distal section 56 can comprise radiopaque markers (e.g., in the form of marker bands around the coils). For yet another example, the strut 55 and certain segments of the distal section 56 can comprise radiopaque markers. For another example, the coils 62 in the distal section 56 can themselves comprise (e.g., be made from) a radiopaque material (e.g., platinum-tungsten alloy). For still another example, certain segments of the proximal section 52 (e.g., the tapered portions 53, tips of peaks) can comprise radiopaque markers. For another example, structural struts in the proximal section 52 can themselves comprise (e.g., be made from) a radiopaque material. FIG. 5 depicts a proximal portion of the distal portion 56 comprising a radiopaque marker 64. The amount and type of radiopaque material used can depend, inter alia, on process technologies, desired level of radiopacity, mechanical properties of the radiopaque material, and corrosion properties of the radiopaque material.

In certain embodiments, the intraluminal device 50 is configured to be positioned near a junction of a bifurcation (e.g., a neurovascular bifurcation (e.g., the basilar tip area)) comprising at least one afferent vessel, efferent vessels, and an aneurysm having a fundus and a neck. For example, in some embodiments, the proximal section 52 is suitably dimensioned to fit in an afferent vessel of a bifurcation (e.g., having a diameter between about 2 mm and about 12 mm, having a diameter between about 6 mm and about 8 mm, having a diameter less than about 15 mm, having a diameter greater than about 1 mm). In some embodiments, the device 50 is configured to treat an aneurysm by providing integrated embolization coils 62 and supporting the embolization coils 62 so that they remain positioned within the aneurysm. In some embodiments, the distal section 56 comprises embolization coils 62 that can be placed within a fundus of an aneurysm in order to treat the aneurysm. In some embodiments, the device 50 comprises an anchor section 52 that can anchor the device 50 in a vessel (e.g., afferent vessel). The anchor or proximal section 52 provides anchoring to the remainder of the device 50, to help maintain the device 50 in a desired position. In some embodiments, the proximal section 52 and the intermediate section 54 bear the weight of the distal section 56. The proximal section 52 and the intermediation section 54 bearing the weight of the distal section 56 can cause the embolization coils 62 to remain within the fundus of the aneurysm and inhibit prolapse of the distal section 56 into afferent and/or efferent vessels. In certain embodiments, the intraluminal device 50 is configured to act as a scaffolding to inhibit or prevent dislodging, herniation, or prolapse of objects (e.g., embolization coils, embolization fluid, thrombi, etc.) out of a neck of an aneurysm. For another example, in some embodiments, the distal section 56 is dense enough that such objects cannot pass (e.g., due to coil packing density). In some embodiments, the distal section 56, while comprising coils, can allow the insertion of additional embolic material therethrough (e.g., through apertures or spaces between each coil, spaces between turns of the coil). In certain embodiments, the device 50 is configured to permit perfusion of fluid (e.g., blood) to efferent vessels of a bifurcation. For yet another example, in some embodiments, the intermediate section 54 is substantially devoid of a covering, mesh, or other material, thereby allowing fluid to flow substantially unimpeded.

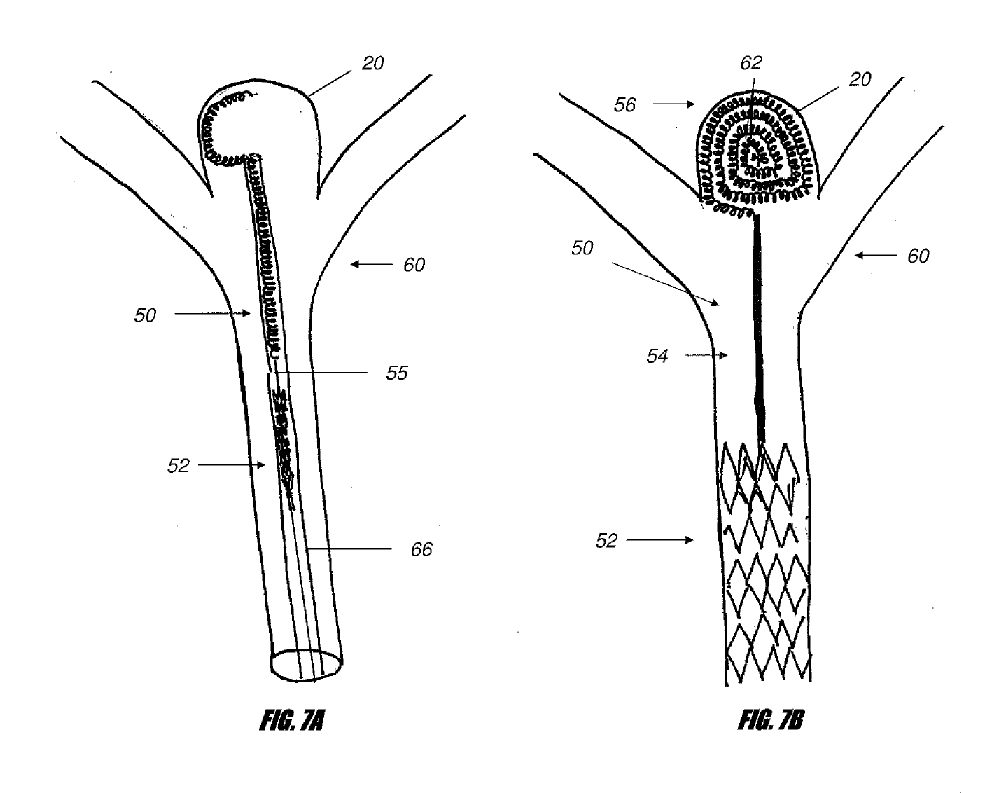

FIGS. 7A and 7B illustrate an example embodiment of a method for treating an aneurysm 20 using the intraluminal device 50 at a confluence of afferent and efferent vessels or “junction” at a bifurcation 60 having an aneurysm 20. In some embodiments, the vessels are neurovascular or cranial. For example, the vasculature can include the basilar tip aneurysm, the middle cerebral artery, the anterior communicating artery, or the internal carotid bifurcation. Treatment of other vasculature, including other than neurovascular or cranial, is also possible.

FIG. 7A shows a delivery catheter 66 within the afferent vessel. The catheter 66 contains part of the intraluminal device 50 in a compressed state. For the sake of clarity, FIG. 7A depicts both the catheter 66 and the device 50 within the catheter (e.g., the view from within the catheter 66). In FIG. 7A, the device 50 is being deployed from the catheter 66 (e.g., by being pushed out with a plunger, by retracting the catheter 66 while the device 50 remains stationary, etc.) and expanding as described herein. In some embodiments, the device 50 comprises a self-expanding and/or a shape-memory material that automatically expands (e.g., self-expands) towards an uncompressed state or does so upon the application of warm fluid (e.g., saline). The strut 55 of the intermediate section 54 allows fluid flow to the efferent vessels. FIG. 7B illustrates the device 50 with the proximal section 52 anchored in the afferent vessel and the distal section 56 in its expanded state within the aneurysm 20. In the embodiment depicted in FIG. 7B, the device has been released and the catheter 66 has been withdrawn from the vasculature.

Intraluminal devices described herein can avoid the use of additional embolic material, for example because the coil 62 in the distal section 56 is sufficient to cause the aneurysm to thrombose. In some embodiments, the distal section 56 is configured to allow insertion of additional embolic material therethrough (e.g., through spacing between coils, through small openings in a 3D configuration) after placement of the intraluminal device 50. For example, in some embodiments, the device 50 comprises a distal section 56 comprising 3D framing coils that can aid in inhibiting objects (e.g., embolization coils, thrombi, etc.) from protruding from the aneurysm 20. After deployment of the device 50, helical embolization coils can be inserted into the aneurysm 20. The option to insert additional embolic material after deployment of the device 50 can advantageously allow for more precise filling of the aneurysm 20. The more precise filling can, at least in part, result from the capability of selecting an embolization material that is most appropriate to fill the remainder of the aneurysm 20 while presenting a low probability of rupture. For example, helical coils are less stiff than 3D framing coils and so inserting helical coils to fill the aneurysm 20 can present less risk of rupture. The additional embolic material can be a single embolization coil, a plurality of embolization coils, and/or other embolic material (e.g., embolic fluid such as Onyx®, available from ev3). In some embodiments, the additional embolic material is inserted in the fundus of the aneurysm 20 using the same catheter 66 from which the device 50 is deployed. In some embodiments, the embolization coils 62 are inserted in the fundus of the aneurysm 20 using a different catheter than the catheter 66 from which the device 50 is deployed. In certain such embodiments, a guidewire can be used to guide both catheters. The coils 62 in the distal end 56 of the device 50 acts as a scaffolding to inhibit or prevent dislodging or prolapse of objects out of the aneurysm 20. The device 50 also allows perfusion of fluid (e.g., blood) from the afferent vessel(s) to the efferent vessel(s). If the position of the device 50 is not as desired, it can be pulled back inside the delivery catheter 66, repositioned, and redeployed at a different (e.g., better) position.

In some embodiments, final release of the intraluminal device 50 is mechanical (e.g., by a release mechanism). In some embodiments, release of the device 50 is electrolytic (e.g., by applying a small current until a proximal tip of the tapered portions 53 corrodes away). In some embodiments, final release of the device 50 is chemical (e.g., by dissolving a connecting portion with a biocompatible solvent such as DMSO). Other detachment mechanisms are also possible. The catheter 66 can then be withdrawn from the bifurcation 60, thereby leaving or permanently positioning the device 50 at the junction of the bifurcation 60.

The term “permanently” does not mean that the intraluminal device 50 is impossible to remove and/or reposition a later time. In some embodiments, the delivery catheter 66 or a different catheter can be used to retrieve or reposition the device 50. In certain embodiments, the device 50 can be retracted into a catheter after being deployed. The device 50 can then be repositioned, for example, at a new rotational position, more proximal or distal to an afferent vessel and/or an efferent vessel, etc, or can be completely removed from the body, for example prior to delivery of a new device (e.g., a different device 50). In some embodiments, only the proximal section 52 or the proximal section 52 and the intermediate section 54 can be retracted into a catheter after being deployed. The proximal section 52 or the proximal section 52 and the intermediate section 54 can then be repositioned and redeployed at a different location or orientation. Once the user is satisfied with the repositioned properties of the device 50 (e.g., size, position, rotation, shape, interaction with the vessels, etc.), the device 50 can be released.

In some embodiments in which the intraluminal device 50 can be electrolytically detached and in which the distal section 56 comprises a different material than the proximal section 52, applying a current can disadvantageously cause corrosion of the intersection between the materials of the proximal section 52 and the distal section 56, and can cause separation of the distal section 56. In certain embodiments, the device 50 comprises an insulating material to inhibit separation of the distal section 56. For example, the different materials of the proximal section 52 and the distal section 56 can be spatially (e.g., longitudinally) separated by an insulating material. For another example, the intersection between the different materials of the proximal section 52 and the distal section 56 can be electrically insulated (e.g., coated). In some embodiments, the intermediate section 54 comprises an electrically insulating material. In some embodiments, a proximal part of the proximal section 52 is electrically isolated from the remainder of the device 50. Other configurations are also possible. For example, in some embodiments, parts or the entirety of the device 50 comprises an electrically insulating coating. In some embodiments, the insulating coating or material comprises a polymer (e.g., parylene, polyethylene, polypropylene, polyurethane, polyethylene terephthalate, etc.). Other materials for the insulating coating or material are also possible.

In some embodiments in which the intraluminal device 50 can be electrolytically detached and in which the distal section 56 comprises a different material than the proximal section 52, applying a current can be utilized to cause corrosion of the intersection between the materials of the proximal section 52 and the distal section 56, and can cause separation of selected portions of the distal section 56. In certain embodiments, the device 50 comprises an insulating material to inhibit complete separation of the distal section 56 as described herein, but allows corrosion for separation of certain parts of the distal section 56. For example, in an embodiment in which the distal section 56 comprises framing coils and filler coils, the framing coils can be insulated and the filler coils by be uninsulated.

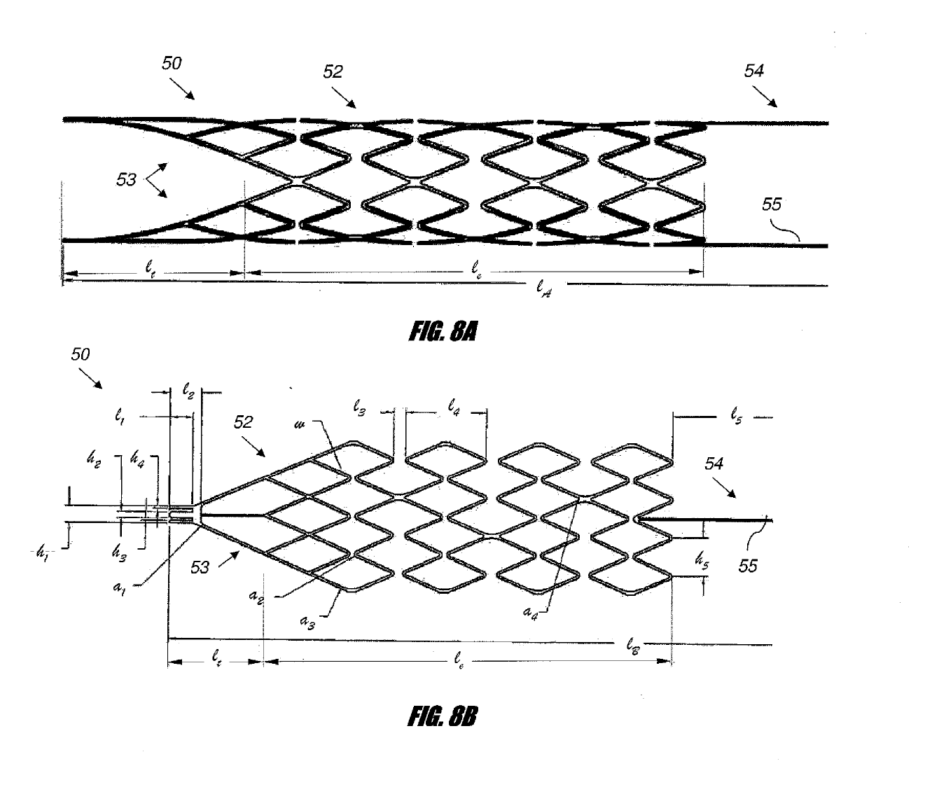

FIGS. 8A and 8B illustrate an example embodiment of the proximal section 52 and intermediate section 54 of a vascular remodeling intraluminal device 50 at a stage of an example manufacturing process comprising cutting and shaping a metallic tube (e.g., a laser cut hypotube), FIG. 8B being rotated 90° with respect to FIG. 8A. A laser can cut out portions of the tube, leaving a plurality of filaments in the proximal section 52 and struts 55 in the intermediate section 54. Other cutting methods (e.g., chemical etch, mechanical cutting, etc.) are also possible.

FIG. 9 illustrates an example embodiment of a vascular remodeling intraluminal device 100 comprising an anchor section 102 and an integrated coil distal section 106. The device 100 can be more compliant than the vasculature in which it is deployed such that it can be somewhat misshapen after being deployed, and that certain shapes described herein are when the device 100 is an expanded state with no restriction. The device 100 comprises a proximal section 102 (or “bottom section” or “main body” or “stem” or “tubular portion” or “anchoring section”), an intermediate section 104 (or “middle section” or “open portion” or “flow section”), and a distal section 106 (or “top section” or “distal portion” or “coil portion” or “integrated coil section” or “treatment section”). The device 100 can be delivered via a catheter (e.g., microcatheter) into a bifurcation to treat an aneurysm with minimal interruption of blood flow in afferent and efferent vessels. In some embodiments, the device 100 can be retrieved and/or repositioned.

The intermediate section 104 comprises a plurality of struts 105. The struts 105 can be straight, curved, or otherwise shaped, such as having design features like the proximal section 102 with the same or a different cell size. The struts 105 couple the proximal section 102 to the distal section 106. The struts 105 can also bias the distal section 106 away from the proximal section 102 (e.g., into an aneurysm). In some embodiments, the struts 105, in conjunction with the proximal section 102, bear the weight of the distal section 106, allowing the distal section 106 to maintain its position within the fundus of an aneurysm. In some embodiments, the struts 105 have a substantially rectangular or flat cross section (e.g., embodiments in which the struts 105 comprise a ribbon or uncut portion of a metallic tube or sheet). In some embodiments, the struts 105 have a substantially round (e.g., circular, elliptical, ovoid) cross section (e.g., embodiments in which the struts 55 comprise round filaments). In some embodiments (e.g., the intraluminal device 100 of FIG. 9), the plurality of struts comprises two struts. In some embodiments, the plurality of struts comprises greater than two struts. In some embodiments, the plurality of struts comprises between about two struts and about twelve struts (e.g., between about three struts and about eight struts, three struts, four struts, five struts, six struts, seven struts, or eight struts). Other numbers of struts are also possible. The number of struts 105 can be selected, for example, based on the expected weight of the integrated coils. For example, as coil weight increases, the number of struts 105 can increase. For another example, the number of struts 105 can be selected based on the number of integrated embolization coils 112 in the distal section 106. Each embolization coil 112 in the distal section 106, for example, can correspond to an individual strut 105. For another example, each of the ends of embolization coil 112 in the distal section 106, for example, can correspond to an individual strut, as depicted in FIG. 9. Free ends of embolization coils can tend to dislodged, and connecting both ends of the embolization coil 112 to a strut 105 can advantageously inhibit ends of the embolization coil 112 from dislodging out of the aneurysm. In certain embodiments, the struts 105 can be equally spaced and/or oriented on opposite sides of the device 100 (e.g., two struts 180° apart along the circumference of the device 100, three struts 120° apart along the circumference of the device 100, four struts 90° apart along the circumference of the device 100, etc.). When the device 100 is placed at a bifurcation, the intermediate section 104 can self-expand and allows perfusion of blood to efferent vessels because the strut 105 does not block fluid flow.

In certain embodiments, the plurality of struts can be integrally fabricated with the proximal section 102 (e.g., by being cut from the same tube) and/or the distal section 106 (e.g., by being an extensions of coils 112). In certain embodiments, the plurality of struts can be made from a different piece and is attached (e.g., welded, glued, adhered, mechanically crimped, mechanically swaged, braided, physical vapor deposited, chemical vapor deposited, etc.) to each of the proximal section 102 and the distal section 106. Separately formed struts 105 allow the struts 105 to be a different material from the proximal section 102 and the distal section 106, although flat pieces of metal can also comprise multiple sections comprising different metals. In some embodiments, the plurality of struts comprises a biocompatible metal and/or biocompatible polymer. In some embodiments, the plurality of struts comprises a radiopaque material (e.g., in the form of a radiopaque core, cladding, coating, small coiled wire, marker band, etc.), which can act as radiopaque markers for improved visibility of the intraluminal device 100 during a procedure and/or following optional implantation.

In some embodiments, the intraluminal device 100 comprises an anchor or proximal section 102 that is flexible and yet has enough radial force to anchor or maintain the position of the device 100 at a bifurcation after deployment (e.g., to inhibit or prevent longitudinal migration of the device 100). In some embodiments, the proximal section 102 has enough radial force to bear the weight of the intermediate section 104 and the distal section 106. In certain embodiments, the proximal section 102 has a first diameter and the distal section 106 has a second diameter greater than the first diameter (e.g., due to expansion of the integrated coils 112 within the aneurysm, etc.), which can cause the strut 105 to be angled or curved outwards from the longitudinal axis defined by the proximal section 102. In certain embodiments, the proximal section 102 has a round (e.g., circular, elliptical, or ovoid) cross section. In some embodiments, the proximal section 102 includes filaments having a substantially rectangular or flat cross section (e.g., embodiments in which the proximal section 102 comprises ribbons or uncut portions of a metallic tube or sheet). In some embodiments, the proximal section 102 includes filaments having a substantially round (e.g., circular, elliptical, ovoid) cross section (e.g., embodiments in which the proximal section 102 comprises round filaments). In some embodiments, the proximal section 102 comprises a combination open cell and closed cell design and coupling struts (e.g., as illustrated in FIG. 9A), described in further detail herein. Other patterns of the proximal section 102 are also possible, for example as described with respect to FIGS. 5 and 11A-11J. In certain such embodiments, the proximal section 102 can achieve good flexibility and/or have good vasculature conformance. In some embodiments, the proximal section 102 comprises a plurality of woven filaments.

When the intraluminal device 100 is placed at a bifurcation, the proximal section 102 provides anchoring of the device 100 in the afferent vessel. The proximal section 102 can also facilitate delivery, positioning, retrieval, and/or repositioning of the device 100. In some embodiments, the proximal end of the proximal section 102 comprises a detachment mechanism. A detachment mechanism at the proximal end of the proximal section 102 allows for permanent placement of the entire device 100. Detachment of the device 100 can be achieved using electrolytic, mechanical, or chemical detachment. Other detachment mechanisms are also possible.

In certain embodiments, the proximal section 102 is fully retrievable back into a catheter, which can allow repositioning of portions of the intraluminal device 100. In certain embodiments, the proximal section 102 and the intermediate section 104 are fully retrievable back into a catheter, which can allow repositioning of portions of the device 100. In certain embodiments, the proximal section 102, the intermediate section 104, and the distal section 106 are fully retrievable back into a catheter, which can allow repositioning of portions (e.g., the entirety) of the device 100.

FIG. 9 illustrates an embodiment in which the proximal end of the proximal section 102 comprises two tapered portions 103. The tapered portions 103 can allow the intraluminal device 100 or portions thereof (e.g., the proximal section 102) to be retrieved back into a catheter. For example, if the device 100 is being pulled into a catheter, the tapered portions 103 can radially compress the proximal section 102.

The distal section 106 can comprise integrated embolization coils 112 that can be placed within the fundus of an aneurysm. The distal section 106 can be atraumatic (e.g., comprising flexible materials, atraumatic shapes, etc.) to inhibit damaging or rupturing aneurysms. The distal section 106 can be self-aligning to accommodate possible misalignment between the afferent vessel and the neck of the aneurysm. The distal section 106 or portions thereof can be self-conforming to irregular contours of the aneurysm.

The distal section 106 comprises embolization coils 112 that can be placed within the fundus of an aneurysm to treat the aneurysm. In some embodiments, the distal section 106 comprises standard helical embolization coils (e.g., coils 62a as depicted in FIG. 6A). In some embodiments, the distal section 106 comprises helical embolization coils with varying diameter and/or pitch (e.g., coils 62b as depicted in FIG. 6B). In some embodiments, the distal section 106 comprises three-dimensional (3D) embolization coils 62c, 62d (e.g., ev3 Axium® Coils), as shown in FIGS. 6C and 6D. Other coil configurations are also possible (e.g., a braided configuration, a twisted configuration, etc.). In some embodiments, the distal section 106 comprises one embolization coil 112. In some embodiments, the distal section 106 comprises two embolization coils 112. Other numbers of embolization coils 112 are also possible (four, five, six, etc.). In some embodiments, the embolization coils 112 comprises a coil diameter of between about 0.04 inches and about 1 inch (approx. between about 1 mm and about 25 mm). In some embodiments, the embolization coils 112 comprise a coil length of between about 0.4 inches and about 20 inches (approx. between about 10 mm and about 510 mm).

Different intraluminal devices 100 can comprise distal section 106 with embolization coils 112 having different properties (e.g., thickness, cross-section or profile, length, packing density, pitch, shape, type, materials, base shape, etc.). For example, the coils 112 of the distal sections 106 of different devices 100 can have a different stiffness, cross-section, flexibility, etc. and combinations thereof. Different shapes, sizes and other properties of the coils 112 can allow for selection of a device 100 from a system of devices 100 that comprises a distal section 106 that is appropriate for the particular aneurysm to be treated (e.g., based on size, shape, etc). For instance, a physician can be able to select a device 100 having a distal section 106 that is most appropriate for the patient and/or vasculature to be treated (e.g., by browsing through a catalogue, by selecting from a kit, etc.). For example, when the aneurysm has a narrow neck, a device 100 comprising a distal section 106 comprising a thin (e.g., coil diameter of between about 0.04 inches and about 1 inches (approx. between about 1 mm and about 25 mm)), more flexible coil 112 can be selected. For another example, when the aneurysm has a wide neck, a device 100 comprising a distal section 106 comprising a stiff, dense coil 112 can be selected. For yet another example, when the aneurysm is small, a device 100 comprising a distal section 106 comprising a short (e.g., coil length of between about 0.4 inches and about 0.6 inches (approx. between about 10 mm and about 15 mm)) and/or compact (e.g., coil diameter of between about 0.04 inches and about 0.5 inches (approx. between about 1 mm and about 13 mm)) coil 112 can be selected. For still yet another example, when the aneurysm is large, a device 100 comprising a distal section 106 comprising a long (e.g., coil length of between about 1 inch and about 15 inches (approx. between about 25 mm and about 380 mm)) and/or voluminous coil (e.g., coil diameter of between about 0.5 inches and about 1 inches (approx. between about 13 mm and about 25 mm)), coil 112 can be selected.

In some embodiments in which the intraluminal device 100 comprises a distal section 106 comprising a plurality of embolization coils 112, the coils 112 in the distal section 106 can have different properties (e.g., thickness, cross-section or profile, length, packing density, pitch, shape, type, materials, base shape etc.). Coils 112 with differing properties can allow for selection of a device 100 comprising a distal section 106 with properties appropriate for filling a particular aneurysm (e.g., based on size and shape). For example, when the device 100 is being used to treat an aneurysm with a wide neck, a device 100 comprising a distal section 106 comprising a stiff, dens coil 112 (e.g., to frame the neck of the aneurysm, to keep objects from protruding from the neck of the aneurysm) in combination with a thin, (e.g., coil diameter of between about 0.04 inches and about 0.5 inches (approx. between about 1 mm and about 13 mm)) flexible, filler coil 112 (e.g., to fill the aneurysm).

In some embodiments, a combination of different types of coils 112 can be used in the distal section 106 of a single intraluminal device 100. For example, with reference to the coils described in FIGS. 6A-6D, a device 100 can comprise a distal section 106 comprising 3D coils 62c and standard helical coils 62a. For another example, a device 100 can comprise 3D helical coils 62d and helical coils of varying diameter and/or pitch 62b. For another example, a device 100 can comprise 3D coils 62c and helical coils of varying diameter and/or pitch 62b. For another example, a device 100 can comprise 3D helical coils 62d and standard helical coils 62a. For another example, a device 100 can comprise standard helical coils 62a and helical coils of varying diameter and/or pitch 62b. For another example, a device 100 can comprise 3D helical coils 62d and 3D coils 62c. For another example, a device 100 can comprise 3D coils 62c, 3D helical coils 62d, and helical coils of varying diameter and/or pitch 62b. For another example, a device 100 can comprise 3D coils 62c, standard helical coils 62a, and helical coils of varying diameter and/or pitch 62b. Other combinations of coils are also possible.

In some embodiments, the distal section 106 comprises embolization coils 112 that are arranged in such a configuration that they provide a frame or basket to inhibit inhibiting the protrusion of objects (e.g., thrombi, coils, etc.) from the aneurysm into the junction or confluence of the bifurcation. For example, in some embodiments, the distal section 106 comprises 3D framing embolization coils 112 that can aid in inhibiting the protrusion of objects (e.g., thrombi, coils, embolization fluid, etc.) from the aneurysm. For another example, in some embodiments, the distal section 106 comprises 3D helical framing embolization coils 112 that can aid in inhibiting the protrusion of objects (e.g., thrombi, coils, embolization fluid, etc.) from the aneurysm.

In some embodiments, the intraluminal device 100 comprises a metallic material (e.g., platinum, tungsten, tantalum, palladium, lead, gold, titanium, silver, etc.). In some embodiments, the device 100 comprises a metal alloy (e.g., platinum alloy (e.g., platinum-tungsten, platinum-iridium), tungsten alloy, stainless steel, tantalum alloy, etc.). In some embodiments, the device 100 comprises a platinum-tungsten alloy (e.g., T10 PtW). In some embodiments, the device 100 comprises a self-expanding, super elastic, and/or a shape-memory material (e.g., comprising Nitinol, CoCr alloy, shape memory polymers (e.g., polyglycolic acid, polylactic acid), etc.), thereby causing the device 100 to be self-expanding under certain conditions (e.g., not restrained by a catheter). In some embodiments, the device 100 comprises a bioabsorbable polymer (e.g., polylactic acid, polyglycolic acid, poly(lactic-co-glycolic acid), poly-epsilon-caprolactone, and/or naturally derived bioabsorbable polymers, etc.), thereby causing the device 100 to bioabsorb over time at a rate dependent on the composition of bioabsorbable polymer(s). In some embodiments, the proximal section 102, the intermediate section 104, and/or the distal section 106 comprises different materials. For example, the distal section 106 can comprise platinum-tungsten alloy while the proximal section 102 and the intermediate section 104 comprise Nitinol. For another example, the distal section 106 can comprise polymer material while the proximal section 102 and the intermediate section 104 comprise metallic material, different polymer material, etc. For yet another example, the distal section 106 can comprise metallic material while the proximal section 102 and the intermediate section 104 comprise different metallic materials, polymer material, etc. Other combinations of the materials described herein and other materials within a single device 100 are also possible.

The intraluminal device 100 can assume a low profile compressed state (e.g., confined within a catheter) for delivery. Upon deployment from the catheter, the device 100 expands (e.g., self-expands) from the compressed state to an expanded state. The device 100 comprises integrated coils 112 in the distal section 106 that can have a compressed or substantially linear configuration when inside the catheter and have a different expanded configuration when deployed.

In some embodiments, the intraluminal device 100 comprises a radiopaque material such as platinum, platinum-iridium, and/or tantalum (e.g., being at least partially formed from the radiopaque material (e.g., having a radiopaque layer, consisting of a radiopaque material), including radiopaque markers). For example, at least some of the plurality of struts can comprise radiopaque markers. For another example, certain segments of the distal section 106 can comprise radiopaque markers (e.g., in the form of marker bands around the integrated coils). For yet another example, some of the struts 105 and certain segments of the distal section 106 can comprise radiopaque markers. For another example, integrated coils 112 in the distal section 106 can themselves comprise (e.g., be made from) a radiopaque material (e.g., platinum-tungsten alloy). For still another example, certain segments of the proximal section 102 (e.g., the tapered portions 103, tips of peaks) can comprise radiopaque markers. For another example, structural struts in the proximal section 102 can themselves comprise (e.g., be made from) a radiopaque material. In some embodiments, a proximal portion of the distal portion 106 comprises a radiopaque marker. The amount and type of radiopaque material used can depend, inter alia, on process technologies, desired level of radiopacity, mechanical properties of the radiopaque material, and corrosion properties of the radiopaque material.

In certain embodiments, the intraluminal device 100 is configured to be positioned near a junction of a bifurcation (e.g., a neurovascular bifurcation (e.g., the basilar tip area)) comprising at least one afferent vessel, efferent vessels, and an aneurysm having a fundus and a neck. For example, in some embodiments, the proximal section 102 is suitably dimensioned to fit in an afferent vessel of a bifurcation (e.g., having a diameter between about 2 mm and about 10 mm, having a diameter between about 1 mm and about 15 mm, having a diameter between about 6 mm and about 8 mm, having a diameter less than about 15 mm, having a diameter greater than about 1 mm). In some embodiments, the device 100 is configured to treat an aneurysm by providing integrated embolization coils 112 and supporting the embolization coils 112 so that they remain positioned within the aneurysm. In some embodiments, the distal section 106 comprises embolization coils 112 that can be placed within a fundus of an aneurysm in order to treat the aneurysm. In some embodiments, the device 100 comprises an anchoring proximal section 102 that can anchor the device 100 in a vessel (e.g., afferent vessel). The proximal section 102 provides anchoring to the remainder of the device 100, to help maintain the device 100 in a desired position. In some embodiments, the proximal section 102 and the intermediate section 104 bear the weight of the distal section 106. The proximal section 102 and the intermediation section 104 bearing the weight of the distal section 106 can cause the embolization coils 112 to remain within the fundus of the aneurysm and inhibit prolapse of the distal section 106 into afferent and/or efferent vessels. In some embodiments, struts 105 of the intermediation section 104 are connected to all free ends of embolization coils 112 in the distal section 106 which can advantageously inhibit any ends of the coils 112 from dislodging out of the aneurysm. In certain embodiments, the device 100 is configured to act as a scaffolding to inhibit or prevent dislodging or prolapse of objects (e.g., embolization coils, embolization fluid, thrombi, etc.) through the neck of an aneurysm. For another example, in some embodiments, the distal section 106 is dense enough that such objects cannot pass (e.g., due to coil packing density). In some embodiments, the distal section 106, while comprising coils, can allow the insertion of other embolic material therethrough (e.g., through apertures or spaces between coils). In certain embodiments, the device 100 is configured to permit perfusion of fluid (e.g., blood) to efferent vessels of a bifurcation. For yet another example, in some embodiments, the intermediate section is substantially devoid of a covering, mesh, thereby allowing fluid to flow substantially unimpeded.

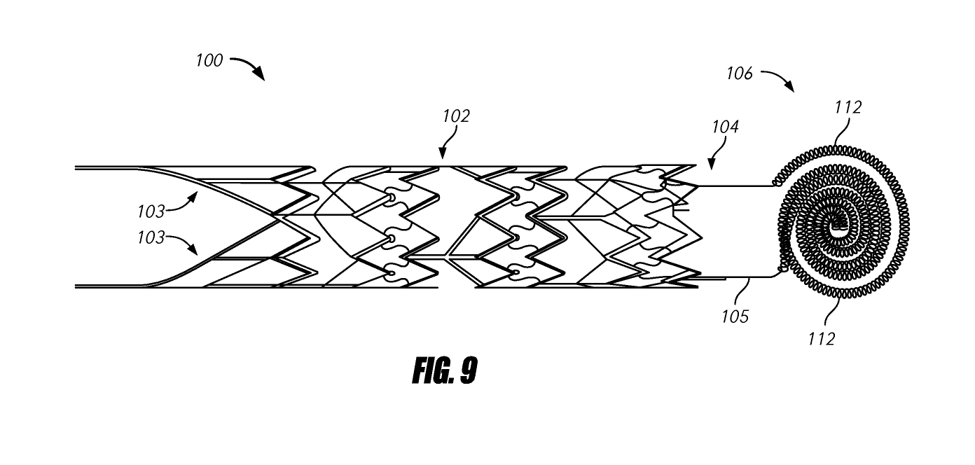

FIG. 10 illustrates an example embodiment of an intraluminal device 100 positioned at a confluence of afferent and efferent vessels or “junction” at a bifurcation having an aneurysm 110. In some embodiments, the vessels are neurovascular or cranial. For example, the vasculature can include the basilar tip aneurysm, the middle cerebral artery, the anterior communicating artery, or the internal carotid bifurcation. In the case of a basilar tip aneurysm, which is near a junction in which the efferent vessels are at about a 90° angle to the afferent vessel, deployment of a conventional aneurysm-bridging stent between the efferent vessels and proximal to the aneurysm neck such that the device can hold embolic material in the aneurysm fundus can be difficult. Treatment of other vasculature, including other than neurovascular or cranial, is also possible.

The proximal section 102 is shown anchored in the afferent or main vessel 116. The intermediate section 104 can allow perfusion to the efferent vessels 114. The distal section 106 is in an expanded state within the aneurysm 110. In some embodiments, positioning of the intraluminal device 100 using the afferent vessel 116 as the delivery path for the device 100 can be accomplished as follows. The distal tip of a delivery catheter (e.g., microcatheter or other catheters that can be tracked through and reach the location of the aneurysm 110) is placed inside the aneurysm 110 or at the neck of the aneurysm 110. The device 100 is then inserted in the proximal end of the catheter or can be positioned in the catheter prior to placement of the distal tip of the delivery catheter. The distal section 106 of the device 100 is then pushed out of the distal end of the catheter (e.g., using a push wire and pulling the catheter back), allowing the distal section 106 to expand (e.g., self-expand) at least partially inside the fundus of the aneurysm 110 (e.g., as illustrated in FIG. 10). The intermediate section 104 of the device 100 is then pushed out of the distal end of the catheter (e.g., using a push wire and pulling the catheter back), allowing the intermediate section 104 to expand (e.g., self-expand) in the junction of the bifurcation. The proximal section 102 of the device 100 is then pushed out of the distal end of the catheter (e.g., using a push wire and pulling the catheter back), allowing the proximal section 102 to expand (e.g., self-expand) in the afferent vessel 116 to maintain the position of the device 100. The device 100 can be fully retrieved inside the catheter, the position of the catheter can be adjusted, and the device 100 can be redeployed, for example to a more desirable position if the position of any section 102, 104, 106 after initial deployment of the device 100 was not as desired after initial deployment. As described herein, in some embodiments, the proximal portion 102 itself or the proximal portion 102 and intermediate portion 104 can be fully retrieved inside the catheter and redeployed, for example to a more desirable position. Additionally or alternatively, the device 100 or the proximal portion 102 or the proximal and intermediate portions 102, 104 can be fully retrieved inside the catheter and a different catheter or the same catheter with a different device (e.g., a device 100 having different dimensions such as diameter of the proximal portion 102, length of the intermediate portion 104, etc.) can be deployed, for example at a more desirable position or with more desirable properties (e.g., better anchoring, better neck coverage, etc.). Once the device 100 is positioned, the device 100 can be detached from the catheter electrolytically, mechanically, or chemically. In such embodiments, detachment can be electrolytic, mechanical, or chemical. The coils 112 in the distal end 106 of the device 100 acts as a scaffolding to inhibit or prevent dislodging or prolapse of objects out of the aneurysm 110. The device 100 also allows perfusion of fluid (e.g., blood) from the afferent vessel(s) to the efferent vessel(s).

In some embodiments in which the intraluminal device 100 can be electrolytically detached and in which the distal section 106 comprises a different material than the proximal section 102, applying a current can disadvantageously cause corrosion of the intersection between the materials of the proximal section 102 and the distal section 106, and can cause separation of the distal section 106. In certain embodiments, the device 100 comprises an insulating material to inhibit separation of the distal section 106. For example, the different materials of the proximal section 102 and the distal section 106 can be spatially (e.g., longitudinally) separated by an insulating material. For another example, the intersection between the different materials of the proximal section 102 and the distal section 106 can be electrically insulated (e.g., coated). In some embodiments, the intermediate section 104 comprises an electrically insulating material. In some embodiments, a proximal part of the proximal section 102 is electrically isolated from the remainder of the device 100. Other configurations are also possible. For example, in some embodiments, parts or the entirety of the device 100 comprises an electrically insulating coating. In some embodiments, the insulating coating or material comprises a polymer (e.g., parylene, polyethylene, polypropylene, polyurethane, polyethylene terephthalate, etc.). Other materials for the insulating coating or material are also possible.

In some embodiments in which the intraluminal device 100 can be electrolytically detached and in which the distal section 106 comprises a different material than the proximal section 102, applying a current can be utilized to cause corrosion of the intersection between the materials of the proximal section 102 and the distal section 106, and can cause separation of selected portions of the distal section 106. In certain embodiments, the device 100 comprises an insulating material to inhibit complete separation of the distal section 106 as described herein, but allows corrosion for separation of certain parts of the distal section 106. For example, in an embodiment in which the distal section 106 comprises framing coils and filler coils, the framing coils can be insulated and the filler coils by be uninsulated.

As described herein, additional embolic material can be placed in the aneurysm 110 before, after, and/or during positioning of the intraluminal device 100. For example, after deployment of the device 100, helical embolization coils can be inserted into the aneurysm 110. The option to insert additional embolic material after deployment of the device 100 can advantageously allow for more precise filling of the aneurysm 110. The more precise filling can, at least in part, result from the capability of selecting an embolization material that is most appropriate to fill the remainder of the aneurysm 110 while presenting a low probability of rupture. For example, helical coils are less stiff than 3D framing coils and so inserting helical coils to fill the aneurysm 110 can present less risk of rupture. The additional embolic material can be a single embolization coil, a plurality of embolization coils, and/or other embolic material (e.g., embolic fluid such as Onyx®, available from ev3). The catheter used to deliver the device 100 or another catheter can be used to deliver additional embolic material into the fundus of the aneurysm 110. In certain such embodiments, a guidewire can be used to guide both catheters. Other delivery methods of the device 100 and other devices described herein are also possible.

FIG. 11 illustrates an example embodiment of a vascular remodeling intraluminal device 150 comprising an anchor section 152 and an integrated coil distal section 156. The intraluminal device 150 can be more compliant than the vasculature in which it is deployed such that it can be somewhat misshapen after being deployed, and that certain shapes described herein are when the device 150 is in an expanded state with no restriction. The device 150 comprises a proximal section 152 (or “bottom section” or “main body” or “stem” or “tubular portion” or “anchoring section”), an intermediate section (or “middle section” or “open portion” or “flow section”), and a distal section 156 (or “top section” or “distal portion” or “coil portion” or “integrated coil section” or “treatment section”). The device 150 can be delivered via a catheter (e.g., microcatheter) into a bifurcation to treat an aneurysm with minimal interruption of blood flow in afferent and efferent vessels. In some embodiments, the device 150 can be retrieved and/or repositioned.

In some embodiments, the intraluminal device 150 comprises an anchor or proximal section 152 that is flexible and yet has enough radial force to anchor or maintain the position of the device 150 at a bifurcation after deployment (e.g., to inhibit or prevent longitudinal migration of the device 150). In some embodiments, the proximal section 152 has enough radial force to bear the weight of the intermediate section 154 and the distal section 156. In certain embodiments, the proximal section 152 has a first diameter and the distal section 156 has a second diameter greater than the first diameter (e.g., due to expansion of the integrated coils 162 within the aneurysm, etc.). In certain embodiments, the proximal section 152 has a round (e.g., circular, elliptical, or ovoid) cross section. In some embodiments, the proximal section 152 includes filaments having a substantially rectangular or flat cross section (e.g., embodiments in which the proximal section 152 comprises ribbons or uncut portions of a metallic tube or sheet). In some embodiments, the proximal section 152 includes filaments having a substantially round (e.g., circular, elliptical, ovoid) cross section (e.g., embodiments in which the proximal section 152 comprises round filaments). In some embodiments, the proximal section 102 comprises a combination open cell and closed cell design and coupling struts (e.g., as illustrated in FIG. 9A), described in further detail herein. Other patterns of the proximal section 152 are also possible, for example as described with respect to FIGS. 5 and 11A-11J. In certain such embodiments, the proximal section 152 can achieve good flexibility and/or have good vasculature conformance. In some embodiments, the proximal section 152 comprises a plurality of woven filaments.

When the intraluminal device 150 is placed at a bifurcation, the proximal section 152 provides anchoring of the device 150 in the afferent vessel. The proximal section 152 can also facilitate delivery, positioning, retrieval, and/or repositioning of the device 150. In some embodiments, the proximal end of the proximal section 152 comprises a detachment mechanism. A detachment mechanism at the proximal end of the proximal section 152 allows for permanent placement of the entire device 150. Detachment of the device 100 can be achieved using electrolytic, mechanical, or chemical detachment. Other detachment mechanisms are also possible.

In certain embodiments, the proximal section 152 is fully retrievable back into a catheter, which can allow repositioning of portions of the intraluminal device 150. In certain embodiments, the proximal section 152 and the intermediate section 154 are fully retrievable back into a catheter, which can allow repositioning of portions of the device 150. In certain embodiments, the proximal section 152, the intermediate section 154, and the distal section 156 are fully retrievable back into a catheter, which can allow repositioning of portions (e.g., the entirety) of the device 150.

FIG. 11 illustrates an embodiment in which the proximal end of the proximal section 152 comprises one tapered portion 153. The tapered portion 153 can allow the intraluminal device 150 or portions thereof (e.g., the proximal section 152) to be retrieved back into a catheter. For example, if the device 150 is being pulled into a catheter, the tapered portion 153 can radially compress the proximal section 152.

The intraluminal device 150 comprises an intermediate section 154. In some embodiments, the intermediate section 154 includes filaments having a substantially rectangular or flat cross section (e.g., embodiments in which the intermediate section 154 comprises ribbons or uncut portions of a metallic tube or sheet). In some embodiments, the intermediate section 154 includes filaments having a substantially round (e.g., circular, elliptical, ovoid) cross section (e.g., embodiments in which the intermediate section 154 comprises round filaments). Various patterns of the intermediate section 154 are also possible, for example as described with respect to FIGS. 5, 9A, and 11A-11J. In some embodiments, the intermediate section 154 comprises a plurality of woven filaments. The intermediate section 154 couples the proximal section 152 to the distal section 156. The intermediate section 154 can also bias the distal section 156 away from the proximal section 152 (e.g., into an aneurysm).

In some embodiments, the intermediate section 154 is an extension of the proximal section 152 (e.g., the proximal section 152 comprises the intermediate section 154) and so the intraluminal device 150 comprises a pattern of filaments (e.g., the same pattern of filaments) throughout the whole of the device except for the distal section 156. In some embodiments, the intermediate section 154 comprises a plurality of z-shaped segments coupled by struts (e.g., as illustrated in FIG. 11). Other patterns of the intermediate section 154 are also possible, for example as described with respect to FIGS. 14A-14J. In some embodiments, the intermediate section 154 has a round (e.g., circular, elliptical, or ovoid) cross-section. In some embodiments, the intermediate section 154 and the proximal section 152 have a substantially similar diameter. In some embodiments, the intermediate section 154 and the proximal section 152 have different diameters. In some embodiments, the intermediate section 154 can have a varying diameter. For example, the filaments of the intermediate section 154 of FIG. 11 extend radially outwardly creating a round bulge 155 in the intermediate section 154. Other shapes in the intermediate section 154 are also possible. For example, the filaments can bulge out in a non-rounded manner towards a point. A bulge 155 in the intermediate section 154 can advantageously be capable of conforming to a junction of a bifurcation comprising an afferent vessel, efferent vessels, and an aneurysm which can enhance the anchoring of the device 150. The intermediate section 154 of the device 150 can couple the proximal section 152 to the distal section 156. Any portion of the filaments or struts of the intermediate section 154 can be coupled to the distal section 156. For example, in the embodiment depicted in FIG. 11, distally extending struts 157 of the intermediate section 154 couple the intermediate section 154 to the coils 162 of the distal section 156. Other connection points are also possible. For example, the coils 162 can also be attached to peaks, valleys, intermediate portions of struts, longitudinally extending struts, etc. In some embodiments, the intermediate section 154, in conjunction with the proximal section 102, bear the weight of the distal section 156, allowing the distal section 156 to maintain its position within the fundus of an aneurysm. When the device 100 is placed at a bifurcation, the intermediate section 104 can expand and allows perfusion of blood to efferent vessels because the strut 105 does not block fluid flow.

In certain embodiments, the intermediate section 154 is integrally fabricated with the proximal section 152 (e.g., by being cut from the same tube) and/or the distal section 156 (e.g., by being extensions of the coils 162). In certain embodiments, the intermediate section 154 is made from a different piece and is attached (e.g., welded, glued, adhered, mechanically crimped, mechanically swaged, braided, physical vapor deposited, chemical vapor deposited, etc.) to each of the proximal section 152 and the distal section 156. An intermediate section 154 comprising a bulge or ball 155 can undergo heat treatment or shape setting to achieve a rounded shape. An intermediate section 154 integrally fabricated with the proximal section 152 and comprising a bulge or ball 155 can undergo more heat treatment or shape setting than the proximal section 152 to achieve a rounded shape. A separately formed intermediate section 154 allows the intermediate section 154 to be a different material from the proximal section 152 and the distal section 156, although flat pieces of metal can also comprise multiple sections comprising different metals. In some embodiments, the intermediate section 154 comprises a biocompatible metal and/or biocompatible polymer. In some embodiments, the intermediate section 154 comprises a radiopaque material (e.g., in the form of a radiopaque core, cladding, coating, small coiled wire, marker band, etc.), which can act as radiopaque markers for improved visibility of the intraluminal device 150 during a procedure and/or following optional implantation.

The distal section 156 can comprise integrated embolization coils 162 that can be placed within the fundus of an aneurysm. The distal section 156 can be atraumatic (e.g., comprising flexible materials, atraumatic shapes, etc.) to inhibit damaging or rupturing aneurysms. The distal section 156 can be self-aligning to accommodate possible misalignment between the afferent vessel and the neck of the aneurysm. The distal section 156 or portions thereof can be self-conforming to irregular contours of the aneurysm.

The distal section 156 comprises embolization coils 162 that can be placed within the fundus of an aneurysm to treat the aneurysm. In some embodiments, the distal section 106 comprises standard helical embolization coils (e.g., coils 62a as depicted in FIG. 6A). In some embodiments, the distal section 156 comprises helical embolization coils with varying diameter and/or pitch (e.g., coils 62b as depicted in FIG. 6B). In some embodiments, the distal section 156 comprises three-dimensional (3D) embolization coils 62c, 62d (e.g., ev3 Axium® Coils), as shown in FIGS. 6C and 6D. Other coil configurations are also possible (e.g., a braided configuration, a twisted configuration, etc.). In some embodiments, the distal section 156 comprises one embolization coil 162. In some embodiments, the distal section 156 comprises two embolization coils 162. Other numbers of embolization coils 162 are also possible (four, five, six, etc.). In some embodiments, the embolization coils 162 comprises a coil diameter of between about 0.04 inches and about 1 inch (approx. between about 1 mm and about 25 mm). In some embodiments, the embolization coils 162 comprise a coil length of between about 0.4 inches and about 20 inches (approx. between about 10 mm and about 510 mm).

Different intraluminal devices 150 can comprise distal sections 156 with embolization coils 162 having different properties (e.g., thickness, cross-section or profile, length, packing density, pitch, shape, type, materials, base shape, etc.). For example, the coils 162 of the distal sections 156 of different devices 150 can have a different stiffness, cross-section, flexibility, etc. and combinations thereof. Different shapes, sizes and other properties of the coils 162 can allow for selection of a device 150 from a system of devices 150 that comprises a distal section 156 that is appropriate for the particular aneurysm to be treated (e.g., based on size, shape, etc). For instance, a physician can be able to select a device 150 having a distal section 156 that is most appropriate for the patient and/or vasculature to be treated (e.g., by browsing through a catalogue, by selecting from a kit, etc.). For example, when the aneurysm has a narrow neck, a device 150 comprising a distal section 156 comprising a thin (e.g., coil diameter of between about 0.04 inches and about 1 inches (approx. between about 1 mm and about 25 mm)), more flexible coil 162 can be selected. For another example, when the aneurysm has a wide neck, a device 150 comprising a distal section 156 comprising a stiff, dense coil 162 can be selected. For yet another example, when the aneurysm is small, a device 150 comprising a distal section 156 comprising a short (e.g., coil length of between about 0.4 inches and about 0.6 inches (approx. between about 10 mm and about 15 mm)) and/or compact (e.g., coil diameter of between about 0.04 inches and about 0.5 inches (approx. between about 10 mm and about 13 mm)) coil 162 can be selected. For still yet another example, when the aneurysm is large, a device 150 comprising a distal section 156 comprising a long (e.g., coil length of between about 1 inch and about 15 inches (approx. between about 25 mm and about 380 mm)) and/or voluminous coil (e.g., coil diameter of between about 0.5 inches and about 1 inches (approx. between about 13 mm and about 25 mm)), coil 162 can be selected.

In some embodiments in which the intraluminal device 150 comprises a distal section 156 comprising a plurality of embolization coils 162, the coils 162 in the distal section 156 can have different properties (e.g., thickness, cross-section or profile, length, packing density, pitch, shape, type, materials, base shape etc.). Coils 162 with differing properties can allow for selection of a device 150 comprising a distal section 156 with properties appropriate for filling a particular aneurysm (e.g., based on size and shape). For example, when the device 150 is being used to treat an aneurysm with a wide neck, a device 150 comprising a distal section 156 comprising a stiff, dens coil 162 (e.g., to frame the neck of the aneurysm, to keep objects from protruding from the neck of the aneurysm) in combination with a thin (e.g., coil diameter of between about 0.04 inches and about 0.5 inches (approx. between about 1 mm and about 13 mm)), flexible coil 162 (e.g., to fill the aneurysm).

In some embodiments, a combination of different types of coils 162 can be used in the distal section 156 of a single intraluminal device 150. For example, with reference to the coils described in FIGS. 6A-6D, a device 150 can comprise a distal section 156 comprising 3D coils 62c and standard helical coils 62a. For another example, a device 150 can comprise 3D helical coils 62d and helical coils of varying diameter and/or pitch 62b. For another example, a device 150 can comprise 3D coils 62c and helical coils of varying diameter and/or pitch 62b. For another example, a device 150 can comprise 3D helical coils 62d and standard helical coils 62a. For another example, a device 150 can comprise standard helical coils 62a and helical coils of varying diameter and/or pitch 62b. For another example, a device 150 can comprise 3D helical coils 62d and 3D coils 62c. For another example, a device 150 can comprise 3D coils 62c, 3D helical coils 62d, and helical coils of varying diameter and/or pitch 62b. For another example, a device 150 can comprise 3D coils 62c, standard helical coils 62a, and helical coils of varying diameter and/or pitch 62b. Other combinations of coils are also possible.

In some embodiments, the distal section 156 comprises embolization coils 162 that are arranged in such a configuration that they provide a frame or basket to inhibit inhibiting the protrusion of objects (e.g., thrombi, coils, etc.) from the aneurysm into the junction or confluence of the bifurcation. For example, in some embodiments, the distal section 156 comprises 3D framing embolization coils 162 that can aid in inhibiting the protrusion of objects (e.g., thrombi, coils, embolization fluid, etc.) from the aneurysm. For another example, in some embodiments, the distal section 156 comprises 3D helical framing embolization coils 162 that can aid in inhibiting the protrusion of objects (e.g., thrombi, coils, embolization fluid, etc.) from the aneurysm.

In some embodiments, the intraluminal device 150 comprises a metallic material (e.g., platinum, tungsten, tantalum, palladium, lead, gold, titanium, silver, etc.). In some embodiments, the device 150 comprises a metal alloy (e.g., platinum alloy (e.g., platinum-tungsten, platinum-iridium), tungsten alloy, stainless steel, tantalum alloy, etc.). In some embodiments, the device 150 comprises a platinum-tungsten alloy (e.g., T10 PtW). In some embodiments, the device 150 comprises a self-expanding, super elastic, and/or a shape-memory material (e.g., comprising Nitinol, CoCr alloy, shape memory polymers (e.g., polyglycolic acid, polylactic acid), etc.), thereby causing the device 150 to be self-expanding under certain conditions (e.g., not restrained by a catheter). In some embodiments, the device 150 comprises a bioabsorbable polymer (e.g., polylactic acid, polyglycolic acid, poly(lactic-co-glycolic acid), poly-epsilon-caprolactone, and/or naturally derived bioabsorbable polymers, etc.), thereby causing the device 150 to bioabsorb over time at a rate dependent on the composition of bioabsorbable polymer(s). In some embodiments, the proximal section 152, the intermediate section 154, and/or the distal section 156 comprise different materials. For example, the distal section 156 can comprise platinum-tungsten alloy while the proximal section 152 and the intermediate section 154 comprise Nitinol. For another example, the distal section 156 can comprise polymer material while the proximal section 152 and the intermediate section 154 comprise metallic material, different polymer material, etc. For yet another example, the distal section 156 can comprise metallic material while the proximal section 152 and the intermediate section 154 comprise different metallic materials, polymer material, etc. Other combinations of the materials described herein and other materials within a single device 150 are also possible.

The intraluminal device 150 can assume a low profile compressed state (e.g., confined within a catheter) for delivery. Upon deployment from the catheter, the device 150 expands (e.g., self-expands) from the compressed state to an expanded state. The device 150 comprises integrated coils 162 in the distal section 156 that can have a compressed or substantially linear configuration when inside the catheter and have a different expanded configuration when deployed.

In some embodiments, the intraluminal device 150 comprises a radiopaque material such as platinum, platinum-iridium, and/or tantalum (e.g., being at least partially formed from the radiopaque material (e.g., having a radiopaque layer, consisting of a radiopaque material), including radiopaque markers). For example, certain portions of the intermediate section 154 can comprise radiopaque markers. For another example, certain segments of the distal section 156 can comprise radiopaque markers (e.g., in the form of marker bands around the integrated coils). For yet another example, certain portions of the intermediate section 154 and certain segments of the distal section 156 can comprise radiopaque markers. For another example, integrated coils 162 in the distal section 156 can themselves comprise (e.g., be made from) a radiopaque material (e.g., platinum-tungsten alloy). For still another example, certain segments of the proximal section 152 (e.g., the tapered portions 153, tips of peaks) can comprise radiopaque markers. For another example, structural struts in the proximal section 152 or the intermediate section 154 can themselves comprise (e.g., be made from) a radiopaque material. In some embodiments, a proximal portion of the distal portion 156 comprises a radiopaque marker. The amount and type of radiopaque material used can depend, inter alia, on process technologies, desired level of radiopacity, mechanical properties of the radiopaque material, and corrosion properties of the radiopaque material.

In certain embodiments, the intraluminal device 150 is configured to be positioned near a junction of a bifurcation (e.g., a neurovascular bifurcation (e.g., the basilar tip area)) comprising at least one afferent vessel, efferent vessels, and an aneurysm having a fundus and a neck. For example, in some embodiments, the proximal section 152 is suitably dimensioned to fit in an afferent vessel of a bifurcation (e.g., having a diameter between about 2 mm and about 10 mm, having a diameter between about 1 mm and about 15 mm, having a diameter between about 6 mm and about 8 mm, having a diameter less than about 15 mm, having a diameter greater than about 1 mm). In some embodiments, the device 150 is configured to treat an aneurysm by providing integrated embolization coils 162 and supporting the embolization coils 162 so that they remain positioned within the aneurysm. In some embodiments, the distal section 156 comprises embolization coils 162 that can be placed within a fundus of an aneurysm in order to treat the aneurysm. In some embodiments, the device 150 comprises an anchoring proximal section 152 that can anchor the device 100 in a vessel (e.g., afferent vessel). The proximal section 152 provides anchoring to the remainder of the device 150, to help maintain the device 150 in a desired position. In some embodiments, the proximal section 152 and the intermediate section 154 bear the weight of the distal section 106. The proximal section 152 and the intermediation section 154 bearing the weight of the distal section 106 can cause the embolization coils 162 to remain within the fundus of the aneurysm and inhibit prolapse of the distal section 156 into afferent and/or efferent vessels. In certain embodiments, the device 150 is configured to act as a scaffolding to inhibit or prevent dislodging or prolapse of objects (e.g., embolization coils, embolization fluid, thrombi, etc.) through the neck of an aneurysm. For another example, in some embodiments, the distal section 156 is dense enough that such objects cannot pass (e.g., due to coil packing density). In some embodiments, the distal section 156, while comprising coils, can allow the insertion of other embolic material therethrough (e.g., through apertures or spaces between coils). In certain embodiments, the device 150 is configured to permit perfusion of fluid (e.g., blood) to efferent vessels of a bifurcation. For yet another example, in some embodiments, the intermediate section is substantially devoid of a covering, mesh, thereby allowing fluid to flow substantially unimpeded.

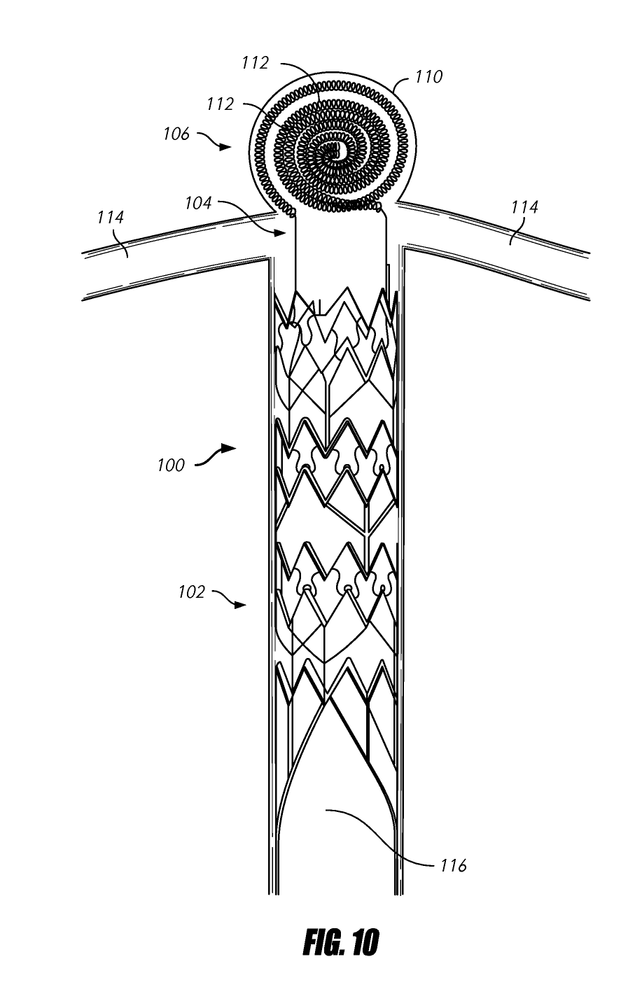

FIG. 12 illustrates an example embodiment of a intraluminal device 150 positioned at a confluence of afferent and efferent vessels or “junction” at a bifurcation having an aneurysm 160. In some embodiments, the vessels are neurovascular or cranial. For example, the vasculature can include the basilar tip aneurysm, the middle cerebral artery, the anterior communicating artery, or the internal carotid bifurcation. In the case of a basilar tip aneurysm, which is near a junction in which the efferent vessels are at about a 90° angle to the afferent vessel, deployment of a conventional aneurysm-bridging stent between the efferent vessels and proximal to the aneurysm neck such that the device can hold embolic material in the aneurysm fundus can be difficult. Treatment of other vasculature, including other than neurovascular or cranial, is also possible.

The proximal section 152 is shown anchored in the afferent or main vessel 158. The intermediate section 154 is shown conforming to the junction of the bifurcation and allowing perfusion to the efferent vessels 164. The distal section 156 is in an expanded state within the aneurysm 160. In some embodiments, positioning of the intraluminal device 150 using the afferent vessel 166 as the delivery path for the device 150 can be accomplished as follows. The distal tip of a delivery catheter (e.g., microcatheter or other catheters that can be tracked through and reach the location of the aneurysm 160) is placed inside the aneurysm 160 or at the neck of the aneurysm 160. The device 150 is then inserted in the proximal end of the catheter or can be positioned in the catheter prior to placement of the distal tip of the delivery catheter. The distal section 156 of the device 150 is then pushed out of the distal end of the catheter (e.g., using a push wire and pulling the catheter back), allowing the distal section 156 to expand (e.g., self-expand) at least partially inside the fundus of the aneurysm 160 (e.g., as illustrated in FIG. 12). The intermediate section 154 of the device 150 is then pushed out of the distal end of the catheter (e.g., using a push wire and pulling the catheter back), allowing the intermediate section 154 to expand (e.g., self-expand) in the junction of the bifurcation. In the embodiment shown in FIGS. 11 and 12, the intermediate section 154 can conform to the shape of the junction of the bifurcation, which can advantageously aid in anchoring the device 150. The proximal section 152 of the device 150 is then pushed out of the distal end of the catheter (e.g., using a push wire and pulling the catheter back), allowing the proximal section 152 to expand (e.g., self-expand) in the afferent vessel 166 to maintain the position of the device 150. The device 150 can be fully retrieved inside the catheter, the position of the catheter can be adjusted, and the device 150 can be redeployed, for example to a more desirable position if the position of any section 152, 154, 156 after initial deployment of the device 150 was not as desired after initial deployment. As described herein, in some embodiments, the proximal portion 152 itself or the proximal portion 152 and intermediate portion 154 can be fully retrieved inside the catheter and redeployed, for example to a more desirable position. Additionally or alternatively, the device 150 or the proximal portion 152 or the proximal and intermediate portions 152, 154 can be fully retrieved inside the catheter and a different catheter or the same catheter with a different device (e.g., a device 150 having different dimensions such as diameter of the proximal portion 152, length of the intermediate portion 154, etc.) can be deployed, for example at a more desirable position or with more desirable properties (e.g., better anchoring, better neck coverage, etc.). Once the device 150 is positioned, the device 150 can be detached from the catheter electrolytically, mechanically, or chemically. In certain such embodiments, detachment can be electrolytic, mechanical, or chemical. The coils 162 in the distal end 156 of the device 150 can, in some embodiments, act as a scaffolding to inhibit or prevent dislodging or prolapse of objects out of the aneurysm 160. For example, the coils 162 can comprise framing coils configured to inhibit or prevent dislodging or prolapse of filler coils out of the aneurysm 160. The device 150 also allows perfusion of fluid (e.g., blood) from the afferent vessel(s) to the efferent vessel(s).

In some embodiments in which the intraluminal device 150 can be electrolytically detached and in which the distal section 156 comprises a different material than the proximal section 152, applying a current can disadvantageously cause corrosion of the intersection between the materials of the proximal section 152 and the distal section 156, and can cause separation of the distal section 156. In certain embodiments, the device 150 comprises an insulating material to inhibit separation of the distal section 156. For example, the different materials of the proximal section 152 and the distal section 156 can be spatially (e.g., longitudinally) separated by an insulating material. For another example, the intersection between the different materials of the proximal section 152 and the distal section 156 can be electrically insulated (e.g., coated). In some embodiments, the intermediate section 154 comprises an electrically insulating material. In some embodiments, a proximal part of the proximal section 152 is electrically isolated from the remainder of the device 150. Other configurations are also possible. For example, in some embodiments, parts or the entirety of the device 150 comprises an electrically insulating coating. In some embodiments, the insulating coating or material comprises a polymer (e.g., parylene, polyethylene, polypropylene, polyurethane, polyethylene terephthalate, etc.). Other materials for the insulating coating or material are also possible.

In some embodiments in which the intraluminal device 150 can be electrolytically detached and in which the distal section 156 comprises a different material than the proximal section 152, applying a current can be utilized to cause corrosion of the intersection between the materials of the proximal section 152 and the distal section 156, and can cause separation of selected portions of the distal section 156. In certain embodiments, the device 150 comprises an insulating material to inhibit complete separation of the distal section 156 as described herein, but allows corrosion for separation of certain parts of the distal section 156. For example, in an embodiment in which the distal section 156 comprises framing coils and filler coils, the framing coils can be insulated and the filler coils by be uninsulated.

As described herein, additional embolic material can be placed in the aneurysm 160 before, after, and/or during positioning of the intraluminal device 150. For example, after deployment of the device 150, helical embolization coils can be inserted into the aneurysm 160. The option to insert additional embolic material after deployment of the device 150 can advantageously allow for more precise filling of the aneurysm 160. The more precise filling can, at least in part, result from the capability of selecting an embolization material that is most appropriate to fill the remainder of the aneurysm 160 while presenting a low probability of rupture. For example, helical coils are less stiff than 3D framing coils and so inserting helical coils to fill the aneurysm 160 can present less risk of rupture. The additional embolic material can be a single embolization coil, a plurality of embolization coils, and/or other embolic material (e.g., embolic fluid such as Onyx®, available from ev3). The catheter used to deliver the device 150 or another catheter can be used to deliver additional embolic material into the fundus of the aneurysm 160. In certain such embodiments, a guidewire can be used to guide both catheters. Other delivery methods of the device 150 and other devices described herein are also possible.

In certain embodiments, sections of the intraluminal device s described herein (e.g., devices 50, 100, 150, combinations of the same, or the like) are integrally fabricated. For example, in some embodiments, the embolization coils (e.g., embolization coils 62, 112, 162) of the distal section (e.g., distal sections 56, 106, 156, combinations of the same, or the like) are integrally fabricated with the struts (e.g., struts 55, 105) or filaments of the intermediate section (e.g., intermediate sections 54, 104, 154, combinations of the same or the like). For example, in the embodiment described with respect to FIGS. 9 and 10, the strut 105 of the intermediate section 104 can be an extension of the wire or filament or other structure forming the embolization coils 112. The wire or filament or other structure forming the embolization coil 112 can be heat set to be in a coil configuration (e.g., as described with respect to FIGS. 6A-6D) in the distal section 106 and can be configured in a strut configuration (e.g., straight, curved, or otherwise shaped) in the intermediate section 104. The strut 105 extending from the embolization coil 112 can be attached (e.g., welded, glued, adhered, mechanically crimped, mechanically swaged, braided, physical vapor deposited, chemical vapor deposited, etc.) to the proximal section 102. For another example, in some embodiments, the intermediate section is integrally fabricated with the proximal section (e.g., being cut from the same tube or sheet). For example, in the embodiment described with respect to FIGS. 9 and 10, the proximal section 102 can be cut from the same tube or sheet as the struts 105. For another example, with reference to the embodiment depicted in FIGS. 11 and 12, the intermediate section 154 and the proximal section 152 can be integrally cut from the same tube or sheet. In certain embodiments, the embolization coils are formed separately from the proximal portion or the proximal and the intermediate portions and are attached (e.g., welded, glued, adhered, mechanically crimped, mechanically swaged, braided, physical vapor deposited, chemical vapor deposited, etc.). In certain such embodiments, the embolization coils can comprise a different material than the proximal section or both the proximal and the intermediate sections. For example, the embolization coils can comprise a platinum-tungsten alloy (e.g., T10 PtW) and the proximal section can comprise Nitinol. For another example, the embolization coils can comprise a platinum-iridium alloy and the proximal section and intermediate section can comprise a bioabsorbable polymer. For another example, the embolization coils and the intermediate section can comprise a platinum-tungsten alloy (e.g., T10 PtW) and the proximal section can comprise a CoCr alloy. Other combinations of materials described herein and otherwise are also possible. Separate or multiple-piece construction can allow for independent selection of materials that are suited for the intended use. In the embodiments described with respect to FIGS. 5-10, some of the struts (e.g., struts 55, 105, and the like) in the intermediate section 104 are integrated with the embolization coils (e.g., being formed from the same coil, wire, filament, etc) and others of the struts are formed separately from the embolization coils and are attached (e.g., welded, glued, adhered, mechanically crimped, mechanically swaged, braided, physical vapor deposited, chemical vapor deposited, etc.). Combination construction can allow easier fabrication than purely multiple-piece construction and also some material selection advantages. The fabrication techniques described herein apply to all devices (e.g., device 50, 100, 150, combinations of the same, and the like) described herein.

FIG. 13 illustrates an example embodiment of the intraluminal device described with respect to FIGS. 9 and 10 at a stage of an example manufacturing process comprising cutting and shaping a metallic sheet. FIG. 13 depicts a proximal section 102 and an intermediate section 104 after having been cut from the sheet. In the embodiment illustrated in FIG. 13, the proximal section 102 and the intermediate section 104 are integrally formed from the metallic sheet and not cut away from each other. A laser or electrochemical etching can cut out portions of the sheet, leaving a plurality of unit cells in the proximal section 102 and strut 105 in the intermediate section 104. Other devices can be fabricated in a similar fashion. For example, with respect to the embodiment described with respect to FIGS. 11 and 12, a laser or electrochemical etching can cut out portions of the sheet, leaving a plurality of unit cells in both the proximal and intermediate sections (e.g., section 152, 154). The cut can be defined by features such as a thickness t of the filaments, effective length lₑ of the proximal section 102, tapered length lₜ of the proximal section 102, and the number of unit cells in the proximal section 102. In some embodiments, the width w is between about 0.02 mm and about 0.2 mm. In some embodiments, the width w is between about 0.03 mm and about 0.1 mm. In some embodiments, the width w is about 0.05 mm. Other widths w are also possible. The width w of the filaments can be uniform throughout the device 100, or can vary depending on location. For example, struts connecting unit cells can be thicker than struts within unit cells. In some embodiments, the length of a unit cell is between about 1 mm and about 7 mm. In some embodiments, the length of a unit cell is between about 2 mm and about 5 mm. Other unit cell lengths are also possible. The dimensions described herein can be uniform throughout the proximal section 102 of the device 100, or can vary depending on location (e.g., increasing from proximal to distal, decreasing from proximal to distal, combinations thereof, and the like). Dimensions can be selected, for example, to accommodate certain vasculature, for flexibility, for wall conformance, etc.

After cutting or chemical etching, the sheet can be reshaped (e.g., into a tube) and the intraluminal device 100 can be heat treated to impart shape setting to at least the proximal section 102. The shape setting process can include several steps comprising, for example, successive shapes using appropriate tooling to stretch and confine the cut sheet into a new shape during the heat treatment. At the end of each heat treatment step, the cut sheet assumes the shape in which it was confined during the heat treatment process. The final shape and size can be obtained by several such steps. For the final shape, there can be a slit along the length of the device 100 (e.g., the opposite sides of the sheet are not joined), or the edge(s) can be welded or otherwise joined together by other methods to form a complete tubular profile. Other devices described herein can also undergo reshaping. For example, the device 150 depicted in FIGS. 11 and 12 can be reshaped to impart a tubular profile to the device 150. A device 150 comprising a bulge or ball 155 in the intermediate section 154 can undergo further heat treatment or shape setting to impart a rounded, bulging shape to the intermediate section 154. Devices described herein can also be formed using a cut metallic tube that is reshaped after being cut, although the properties of the initial tube and the pattern of the cut can be different.

In some embodiments, the distal section 106 of the intraluminal device can be formed separately and be attached (e.g., welded, glued, adhered, mechanically crimped, mechanically swaged, braided, physical vapor deposited, chemical vapor deposited, etc.) to the intermediate section 104. In such embodiments, the embolization coil 112 can go through a shape setting process to achieve a final shape. The shape setting process can start with the coil 112 in the form of a wire (or ribbon or filament). The wire can undergo a first heat treatment or treatments to achieve a first shape (e.g., a helical shape). The helically shaped wire can then undergo a second heat treatment or treatments to achieve a more complex three dimensional shape (e.g., as shown in FIG. 6D). More or fewer heat treatments can be applied to achieve a desired coil configuration. Other initial configurations for the coil 112 are also possible (e.g., ribbons, filaments, etc.). The embolization coil 112 can be attached to the intermediate section 104 of the device 100 before, after, or during undergoing shape setting treatments. Embodiments of the device 100 in which the distal section 106 is integrally formed with the intermediate section 104 can undergo similar shape setting treatments. For example, a wire can undergo a heat treatment to achieve a helical shape in the distal section 106. It can also undergo heat treatment to achieve an elongated shape in the intermediate section 104. The fabrication techniques described herein apply to all devices (e.g., device 50, 150, combinations of the same, and the like) described herein.

FIGS. 14A-14J illustrate example embodiments of proximal sections 1221, 1222, 1223, 1224, 1225, 1226, 1227, 1228, 1229, 1230 that can be incorporated into the intraluminal devices described herein. FIG. 14A illustrates an example embodiment of a proximal section 1221 having an “open cell” design, identifiable by the reverse free-peaks 124 and the forward free-peaks 125. Open cell designs generally provide good flexibility and wall apposition, but can be difficult to retrieve, for example due to reverse free-peaks snagging or catching on the catheter during retrieval. FIG. 14B illustrates an example embodiment of a proximal section 1222 having a “closed cell” design, identifiable by the lack of any peaks due to contact of all cells at intersections 126. FIG. 14C illustrates another example embodiment of a proximal section 1223 having a “closed cell” design, identifiable by the lack of reverse free-peaks 127 and forward free-peaks 128, which are connected by struts 129. Closed cell designs are generally easy to deliver and to retrieve, but can be stiff and provide poor wall apposition (e.g., being prone to kinking rather than bending).

A hybrid of open cell and closed cell designs can advantageously incorporate the advantages of each design and can avoid the potential drawbacks of each design. FIGS. 14D-14H illustrate example embodiments of proximal sections that are “hybrid” or “combination” designs including features of open cell designs and features of closed cell designs. FIG. 14D illustrates an example embodiment of a proximal section 1224 having a hybrid cell design. The proximal section 1224 comprises forward connected peaks 131, 133, forward free-peaks 132, and reverse connected peaks 134. The forward peaks 133 are connected to the next unit cell. The proximal section 1224 does not include any reverse free-peaks (see element 124 of FIG. 14A). FIG. 14E illustrates an example embodiment of a proximal section 1225 having a hybrid cell design. The proximal section 1225 comprises forward connected peaks 131, 133, forward free-peaks 132, and reverse connected peaks 134. The forward peaks 133 are connected to the next unit cell. The proximal section 1225 does not include any reverse free-peaks (see element 124 of FIG. 14A). FIG. 14F illustrates an example embodiment of a proximal section 1226 having a hybrid cell design. The proximal section 1226 comprises forward connected peaks 131, forward free-peaks 132, and reverse connected peaks 134. The proximal section 1226 further comprises valleys 135 connected to the next unit cell. The proximal section 1226 does not include any reverse free-peaks (see element 124 of FIG. 14A). FIG. 14G illustrates an example embodiment of a proximal section 1227 having a hybrid cell design. The proximal section 1227 comprises forward connected peaks 131, forward free-peaks 132, and reverse connected peaks 134. The proximal section 1227 further comprises valleys 135 connected to the next unit cell. The proximal section 1227 does not include any reverse free-peaks (see element 124 of FIG. 14A).

FIG. 14H illustrates an example embodiment of a proximal section 1228 having a hybrid cell design. The proximal section 1228 comprises forward connected peaks 133, forward free-peaks 132, and reverse connected peaks 134. The forward peaks 133 are connected to the next unit cell. Each unit cell comprises forward connected peaks 133 alternating with forward free-peaks 132. The proximal section 1228 further comprises peaks connected to the next unit cell. The proximal section 1228 does not include any reverse free-peaks (see element 124 of FIG. 14A). FIG. 14I illustrates an example embodiment of a proximal section 1229 having a hybrid cell design. The proximal section 1229 comprises forward connected peaks 133, forward free-peaks 132, and reverse connected peaks 134. The forward peaks 133 are connected to the next unit cell. Each unit cell comprises forward connected peaks 133 alternating with forward free-peaks 132. The proximal section 1229 further comprises peaks connected to the next unit cell. The proximal section 1229 does not include any reverse free-peaks (see element 124 of FIG. 14A). In contrast to the proximal section 1228 of FIG. 14H, the proximal section 1229 of FIG. 14I has fewer diagonal struts (e.g., missing in the area 138), which can provide better flexibility and/or wall apposition. FIG. 14J illustrates an example embodiment of a proximal section 1230 having a hybrid cell design. The proximal section 1230 comprises forward connected peaks 133, forward free-peaks 132, and reverse connected peaks 134. The forward peaks 133 are connected to the next unit cell. Each unit cell comprises forward connected peaks 133 alternating with forward free-peaks 132. The proximal section 1230 further comprises peaks connected to the next unit cell. The proximal section 1230 does not include any reverse free-peaks (see element 124 of FIG. 14A). In contrast to the proximal section 1229 of FIG. 14I, the proximal section 1230 of FIG. 147 has straight struts 1391, which can be less prone to twisting during compaction. Combinations of the features of the cell patterns illustrated in FIGS. 14A-14I can be selected based on desired properties of the proximal section.

FIGS. 14B, 14D, and 14F illustrate proximal sections 1222, 1224, 1226, respectively, having one tapered section 123, while FIGS. 14A, 14C, 14E, 14G, 14H, 14I, and 14J illustrate proximal portions 1221, 1223, 1225, 1227, 1228, 1229, 1230, respectively, having two tapered sections 123. A single tapered section 123 can advantageously have only one detachment zone and be easy to release, while a plurality of tapered sections 123 can comprise a detachment zone proximal to each tapered section 123 and can be more difficult to release. A plurality of tapered sections 123 can have a shorter taper length lₜ and a longer effective length lₑ (FIGS. 8A, 8B, and 13), while a single tapered section 123 can have a longer taper length lₜ and a shorter effective length lₑ (FIGS. 8A, 8B, and 13) and can provide less anchoring in the afferent vessel. A plurality of tapered sections 123 can be more symmetrical and provide more uniform wall apposition. A plurality of tapered sections 123 can have less of a tension effect on the vessel, which can result from a single long tapered area applying force to a single side of the vessel. The effective length lₑ of the proximal section can be based on the intended anatomy. Longer lengths can be appropriate for more vessel wall apposition, while shorter lengths can be appropriate for traversing more tortuous anatomy. In some embodiments, the effective length lₑ of the proximal section is between about 5 mm and about 40 mm. In some embodiments, the effective length lₑ of the proximal section is between about 10 mm and about 30 mm. In some embodiments, the effective length lₑ of the proximal section is between about 10 mm and about 20 mm. Other effective lengths lₑ are also possible.

FIGS. 14C, 14F, and 14G illustrate proximal sections 1223, 1226, 1227, respectively, comprising s-shaped struts 129 connecting certain forward peaks and reverse peaks. FIGS. 14D, 14E, and 14J illustrate proximal portions 1224, 1225, 1230, respectively, comprising straight struts 1391 connecting certain forward peaks and reverse peaks. FIGS. 14H and 14I illustrate proximal portions 1228, 1229 comprising c-shaped struts 1392 connecting certain forward peaks and reverse peaks. Connection struts having an s-shape or c-shape can be more flexible, but can be prone to twisting during compaction, while straight struts can be easier to compress but less flexible, which can be acceptable for hybrid cell designs already having suitable flexibility.

FIGS. 14D and 14E illustrate proximal sections 1224, 1225 having tip-to-tip connections between forward and reverse peaks, which can provide a smaller compaction profile. FIGS. 14F, 14G, 14H, and 14I illustrate proximal sections 1226, 1227, 1228, 1229 having at least partially offset tip-to-tip connections between forward and reverse peaks, which can provide increased flexibility and/or can increase vessel conformance.

FIGS. 14D, 14E, 14H, 14I, and 14J illustrate proximal sections 1224, 1225, 1228, 1229, 1230, respectively, having tip-to-tip connections between forward and reverse peaks of unit cells, which can provide an easier compaction profile. FIGS. 14F and 14G illustrate proximal sections 1226, 1227 having valley-to-tip connections between forward and reverse peaks of unit cells, which can provide good flexibility.

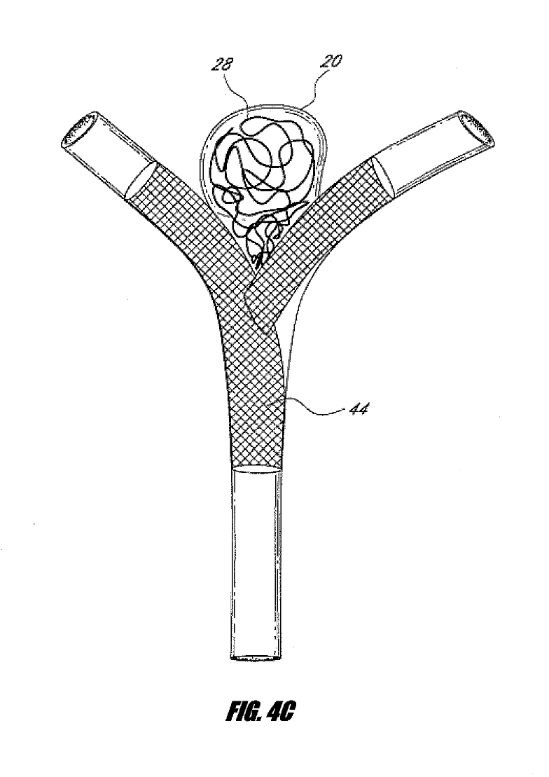

The patterns described herein can be repeated (e.g., repetition of rows of unit cells), adjusted (e.g., different angles, different lengths, different thicknesses, etc.), and/or combined (e.g., permutations of any of the features disclosed herein) based on the desired properties of the proximal section. In some embodiments, the proximal section can be flow diverting, which can allow the intraluminal device to be used across sidewall aneurysms, for example as shown in FIG. 4A. In some embodiments, radiopaque markers are integrated into a portion (e.g., the distal peaks of the forward free-peaks, around the struts, etc.) of the proximal section that the user (e.g., physician) can use to monitor placement of the device.

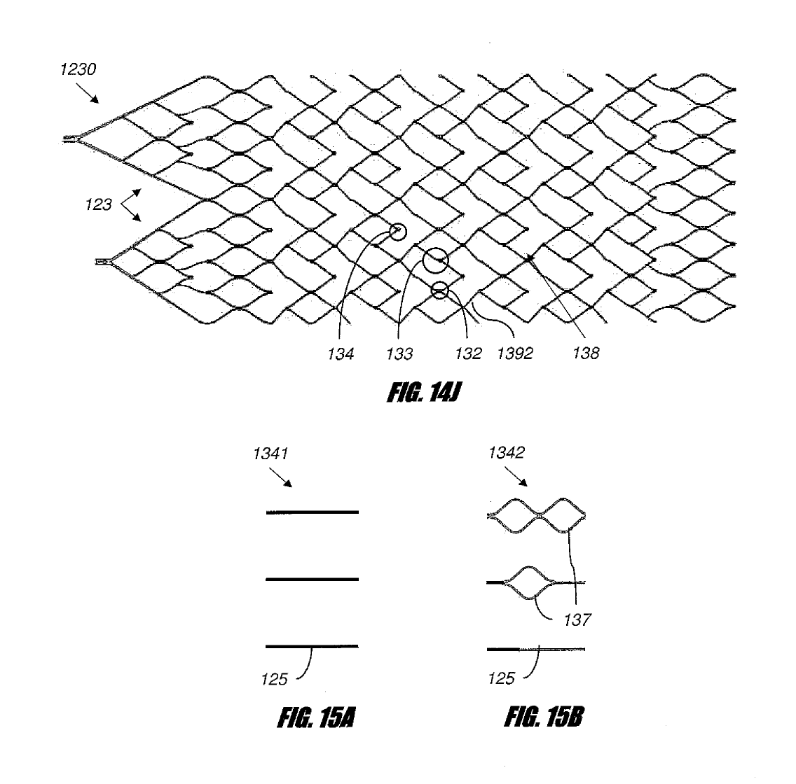

FIGS. 15A and 15B illustrate example embodiments of intermediate sections 1341, 1342 that can be incorporated into the intraluminal devices described herein (e.g., into intraluminal devices 50, 100, combinations of the same, and the like). FIG. 15A illustrates an example embodiment of an intermediate section 1341 comprising a plurality of straight struts. The number of struts can be selected, for example, based on the expected weight of the embolic coils. For example, as coil weight increases, the number of struts can increase. For another example, the number of struts 25 can be selected based on the number of the embolization coils. Each embolization coil, for example, can correspond to an individual strut 25, an end thereof, etc. In some embodiments, the intermediate section 1341 can comprise one strut 125. In some embodiments, the intermediate section 1341 can comprise a plurality of struts 125. In some embodiments, the plurality of struts comprises two struts. In some embodiments, the plurality of struts comprises greater than two struts. In some embodiments, the plurality of struts comprises three struts (e.g., as illustrated in FIG. 15A). In some embodiments, the plurality of struts comprises between about two struts and about twelve struts (e.g., between about three struts and about eight struts, three struts, four struts, five struts, six struts, seven struts, or eight struts). Other numbers of struts are also possible. In certain embodiments, the struts can be equally spaced and/or oriented on opposite sides of the device (e.g., two struts 180° apart along the circumference of the device, three struts 120° apart along the circumference of the device, four struts 90° apart along the circumference of the device, etc.).

FIG. 15B illustrates an example embodiment of an intermediate section 1342 comprising a straight strut and two elongation struts 137 comprising openings. During compaction, the openings of the elongation struts 137 can collapse, thereby increasing the length of the elongation struts 137. In the embodiment illustrated in FIG. 15B, upon compaction the straight strut would maintain length, the middle elongation strut 137 would increase in length somewhat, and the top elongation strut 137 would increase in length the most. The portions of the distal section attached to the strut and elongation struts would be differentiated, which can provide a good compaction profile.

Any combination or permutation of the proximal, intermediate, and distal sections described herein, whether in FIGS. 14A-15B or elsewhere can be used in an intraluminal device for aneurysm treatment or other uses. For example, referring again to FIG. 5, the proximal section 52 is the proximal section 1221 of FIG. 14A, the intermediate section 54 is one strut 55, and the distal section 56 includes a coil 62 that is similar to the embolization coil 62d of FIG. 6D. For another example, referring again to FIG. 9, the proximal section 102 is the proximal section 1227 of FIG. 12G, the intermediate section 54 is a plurality of struts 105, and the distal section 106 includes a plurality of coils 112 that are similar to the embolization coil 62d of FIG. 6D. A large number of permutations are possible by selecting a proximal section from amongst FIGS. 14A-14J (or equivalents or modifications thereof), selecting an intermediate section from amongst FIGS. 14A and 12B (or equivalents or modifications thereof), selecting a distal section from amongst FIGS. 5, 6A-6D, 9, or otherwise as described herein (or equivalents or modifications thereof). Thus, the devices disclosed herein are not limited to any explicitly illustrated embodiment.

As described herein, the proximal section, the intermediate section, and the distal section can be integrally formed from the metallic tube or sheet and not cut away from each other. In embodiments in which all sections of the intraluminal device are integrally fabricated by being cut from the same tube or sheet, the device is of single-piece construction. Single-piece construction can allow for easier manufacturing. Certain portions of the proximal section, the intermediate section, and the distal section can be formed separately. For example, a proximal end segments can be cut from a tube or a sheet and then coupled (e.g., welded, glued, adhered, mechanically crimped, mechanically swaged, braided, physical vapor deposited, chemical vapor deposited, etc.) by connectors. In some embodiments, some or all of the proximal section, the intermediate section, and the distal section can be formed separately, and the parts coupled together (e.g., by being welded, glued, adhered, mechanically crimped, mechanically swaged, braided, physical vapor deposited, chemical vapor deposited, etc.). For example, the proximal section and the intermediate section can be cut from a tube or a sheet and then coupled (e.g., welded, glued, adhered, mechanically crimped, mechanically swaged, braided, physical vapor deposited, chemical vapor deposited, etc.) to the distal section. In certain such embodiments, the distal section can comprise different material than the proximal section and the intermediate section. Combination construction can allow easier fabrication than purely multiple-piece construction and also some material selection advantages.

Referring again to FIGS. 8A, 8B, and 9 but also applicable to FIGS. 14A-15B, the cut can be defined by features such as filament width w, lengths l₁ (e.g., length of a proximal end finger), l₂ (e.g., length of a proximal end segment including fingers), l₃ (e.g., length of a connector coupling proximal section unit cells, length between proximal section unit cells), l₄ (e.g., length of a proximal section unit cell, length of a proximal section unit cell portion), l₅ (e.g., length of intermediate section, length between proximal section and distal section), l₆ (e.g., length between distal section inward-facing peaks), l₇ (e.g., length of the distal section in a partially expanded state), heights h₁ (e.g., height of proximal end segment including fingers), h₂ (e.g., height of a proximal end finger in a first dimension), h₃ (e.g., height between proximal end fingers), h₄ (e.g., height of a proximal end finger in a second dimension), h₅ (e.g., height between free peaks), h₆ (e.g., height of distal section in the expanded state), and angles a₁ (e.g., angle of taper), a₂ (e.g., angle of reverse free peak, angle of reverse connected peaks), a₃ (e.g., angle of at least partially longitudinally projecting filaments), a₄ (e.g., angle of forward free peaks, angle of forward connected peaks), and a₅ (e.g., angle of distal end forward peaks). For different patterns, the configuration and dimensions of certain features will also be different. For example, some cuts can not include certain of the dimensions described herein.

In some embodiments, the width w is between about 0.02 mm and about 0.2 mm. In some embodiments, the width w is between about 0.03 mm and about 0.1 mm. In some embodiments, the width w is about 0.05 mm. Other widths w are also possible. The width w of the filaments can be uniform throughout the intraluminal device 100, or can vary depending on location. For example, struts connecting unit cells can be thicker than struts within unit cells.

In some embodiments, the tapered length lₜ is between about 1.5 mm and about 20 mm. In some embodiments, the tapered length lₜ is between about 4 mm and about 15 mm. Other tapered lengths lₜ are also possible. In some embodiments, the effective length lₑ is between about 5 mm and about 40 mm. In some embodiments, the effective length lₑ is between about 10 mm and about 30 mm. In some embodiments, the effective length lₑ is between about 10 mm and about 20 mm. Other effective lengths lₑ are also possible.

In some embodiments, the length l₂ is between about 0.01 mm and about 2 mm. In some embodiments, the length l₂ is between about 0.05 mm and about 0.75 mm. Other lengths l₂ are also possible. In some embodiments, the length l₃ is between about 0.01 mm and about 3 mm. In some embodiments, the length l₃ is between about 0.1 mm and about 0.5 mm. Other lengths l₃ are also possible. In some embodiments, the length l₄ is between about 1 mm and about 7 mm. In some embodiments, the length l₄ is between about 2 mm and about 5 mm. Other lengths l₄ are also possible. In some embodiments, the length l₅ is between about 0 mm and about 8 mm. In some embodiments, the length l₅ is between about 0 mm and about 10 mm. In some embodiments, the length l₅ is between about 0 mm and about 6 mm. In some embodiments, the length l₅ is between about 6 mm and about 10 mm. In some embodiments, the length l₅ is about 8 mm. In some embodiments, the length l₅ is between about 0 mm and about 5 mm. Other lengths l₅ are also possible. When the length l₅ is 0 mm, the intraluminal device can comprise an proximal section 152 comprising an intermediate section 154, for example as illustrated in FIG. 11. In some embodiments, the length l₆ is between about 0.01 mm and about 3 mm. In some embodiments, the length l₆ is between about 0.05 mm and about 0.5 mm. Other lengths l₆ are also possible. In some embodiments, the length l₇ is between about 0.5 mm and about 10 mm. In some embodiments, the length l₇ is between about 1.5 mm and about 6 mm. Other lengths l₇ are also possible.

In some embodiments, the height h₁ is between about 0.01 mm and about 0.75 mm. In some embodiments, the height h₁ is between about 0.01 mm and about 0.5 mm. Other heights h₁ are also possible. In some embodiments, the height h₄ is between about 0.01 mm and about 0.25 mm. In some embodiments, the height h₄ is between about 0.01 mm and about 0.1 mm. Other heights h₄ are also possible. In some embodiments, the height h₅ is between about 0.25 mm and about 6 mm. In some embodiments, the height h₅ is between about 0.5 mm and about 3 mm. Other heights h₅ are also possible. In some embodiments, the height h₆ is between about 1.5 mm and about 6 mm in the expanded state.

The dimensions described herein, including for example dimensions described with respect to FIG. 8A, can be uniform throughout the proximal section 102 of the intraluminal device 100, or can vary depending on location (e.g., increasing from proximal to distal, decreasing from proximal to distal, combinations thereof, and the like). Dimensions can be selected, for example, to accommodate certain vasculature, for flexibility, for wall conformance, etc. In some embodiments, a reduced number of the connectors coupling proximal end segments can increase the flexibility of the proximal section of the device.

As described herein (e.g., with respect o FIG. 13), after cutting the tube or the sheet, the intraluminal device can be reshaped and the device can be heat treated to impart shape setting to at least the distal section and, at least for a sheet, the proximal section 122. The shape setting process can include several steps comprising, for example, successively shapes using appropriate tooling to stretch and confine the cut tube into a new shape during the heat treatment. At the end of the each heat treatment step, the cut tube or sheet assumes the shape in which it was confined during the heat treatment process. The final shape and size can obtained by several such steps. In some embodiments in which a cut sheet is rolled to form a tube, there can be a slit along the length of the device (e.g., the opposite sides of the sheet are not joined), or the edge(s) can be welded or otherwise joined together by other methods to form a complete tubular profile. In certain such embodiments, the sides can be in contact or can be spaced.

Certain intraluminal devices described herein can be advantageously used to treat aneurysms having a neck ratio (a ratio of fundus width to neck width) greater than about 2 to 1 and/or a neck width greater than about 4 mm. In treatment of such aneurysms, embolization coils can be prone to dislodging into parent vessels because the size and/or shape of the aneurysm is not conducive to maintaining the coils in their inserted locus. The proximal and intermediate sections of the intraluminal device described herein can advantageously bear the weight of the coils and keep them positioned within the fundus of the aneurysm.

Although these inventions have been disclosed in the context of certain embodiments and examples, it will be understood by those skilled in the art that the inventions extend beyond the specifically disclosed embodiments to other alternative embodiments and/or uses of the inventions and obvious modifications and equivalents thereof. In addition, while several variations of the embodiments of the inventions have been shown and described in detail, other modifications, which are within the scope of these inventions, will be readily apparent to those of skill in the art based upon this disclosure. It is also contemplated that various combinations or sub-combinations of the specific features and aspects of the embodiments can be made and still fall within the scope of the inventions. It should be understood that various features and aspects of the disclosed embodiments can be combined with, or substituted for, one another in order to form varying modes of the embodiments of the disclosed inventions. Thus, it is intended that the scope of the inventions herein disclosed should not be limited by the particular embodiments described above.

What is claimed:

1. An intraluminal device comprising:
a proximal section configured to anchor in an afferent vessel;
an intermediate section configured to allow perfusion to efferent vessels; and
a distal section comprising an embolization coil coupled to and extending distally from the intermediate section and configured to be positioned within an aneurism.

2. The device of claim 1, wherein the intermediate section comprises a plurality of struts.

3. The device of claim 1, wherein the embolization coil comprises a standard helical coil.

4. The device of claim 1, wherein the embolization coil comprises a 3D coil.

5. The device of claim 1, wherein the distal section comprises a plurality of embolization coils.

6. The device of claim 1, wherein the proximal section comprises a first material and the distal section comprises a second material different from the first material.

7. The device of claim 6, wherein the first material is insulated from the second material.

8. A method of treating an aneurysm near a junction of a bifurcation having an afferent vessel and efferent vessels, the aneurysm having a neck and a fundus, the method comprising:
advancing a first catheter proximate to the junction of the bifurcation, the catheter at least partially containing a device in a compressed state, the device including:
a proximal section configured to anchor in an afferent vessel;
an intermediate section configured to allow perfusion to efferent vessels; and
a distal section comprising an embolization coil coupled to and extending distally from the intermediate section and configured to be positioned within an aneurism;
deploying the device from at least partially inside the first catheter to outside the first catheter at the junction of the bifurcation, wherein, during deployment,
the distal section self-expands within the fundus of the aneurysm;
the intermediate section self-expands and allows perfusion to the efferent vessels; and
the proximal section self-expands to anchor in an expanded state to the walls of the afferent vessel.

9. The method of claim 8, wherein deploying the device comprises initially deploying the device, retrieving at least a section of the device at least partially back into the first catheter, and redeploying the device.

10. The method of claim 8, further comprising inserting additional embolic material into the aneurysm.

11. The method of claim 10, wherein inserting the additional embolic material comprises deploying the additional embolic material from the first catheter.

12. The method of claim 10, wherein inserting the additional embolic material is before deploying the device.

13. The method of claim 10, wherein inserting the additional embolic material is after deploying the device.

14. A system for treating aneurysms comprising:
at least first and second intraluminal devices, each of the first and second devices comprising:
a proximal section configured to anchor in an afferent vessel;
an intermediate section configured to allow perfusion to efferent vessels; and
a distal section comprising an embolization coil coupled to and extending distally from the intermediate section and configured to be positioned within an aneurism;
wherein the distal section of the first device comprises at least one property that is different from a corresponding property of the second device.

15. The system of claim 14, wherein the first device of the system comprises a distal section comprising a standard helical embolization coil and the second device of the system comprises a distal section comprising a 3D embolization coil.

16. The system of claim 14, wherein the first device of the system comprises a distal section comprising an embolization coil of a first length and the second device of the system comprises a distal section comprising an embolization coil of a second length, the second length being greater than the first length.

17. The system of claim 14, wherein the embolization coil of the first device comprises a first packing density and the embolization coil of the second device comprises a second packing density greater than the first packing density.

18. The system of claim 14, wherein the embolization coil of the first device comprises a first cross-sectional dimension and the embolization coil of the second device has a second cross-sectional dimension greater than the first cross-sectional dimension.

19. The system of claim 14, wherein the distal section of the first device comprises a first number of embolization coils and the distal section of the second device comprises a second number of embolization coils greater than the first number.

20. The system of claim 14, wherein the embolization coil of the distal section of the first device has a first flexibility and the embolization coil of the distal section of the second device has a second flexibility that is less than the first flexibility.
